(19) United States
(12) Patent Application Publication
(10) Pub. No.: US 2024/0085421 A1
(43) Pub. Date: Mar. 14, 2024

(54) METHODS FOR THE IDENTIFICATION OF DEGRONS

(71) Applicant: MONTE ROSA THERAPEUTICS, INC., Boston, MA (US)

(72) Inventors: Richard David Bunker, Basel (CH); Gerald Gavory, Brinckheim (FR); Oliver Horlacher, Olten (CH)

(21) Appl. No.: 18/271,887

(22) PCT Filed: Jan. 13, 2022
(86) PCT No.: PCT/IB2022/050276

§ 371 Date: Jul. 12, 2023

Related U.S. Application Data
(60) Provisional application No. 63/137,082, filed on Jan. 13, 2021.

(51) Int. Cl. G01N 33/573; G01N 33/68

(52) U.S. Cl. CPC G01N 33/573; G01N 33/6845; G01N 2333/9108; G01N 2500/20

(57) ABSTRACT

Provided herein are various methods for identifying candidate substrates for the E3 ligase machinery.

### CLAIM OF PRIORITY

This application claims the benefit of U.S. Provisional Application Ser. No. 63/137,082, filed 13 Jan. 2021. The entire contents of the foregoing are incorporated herein by reference.

### TECHNICAL FIELD

The present disclosure relates to the field of protein degradation. Provided herein are, among other things, methods for the identification of proteins capable of being targeted for degradation by the E3 ligase machinery.

### BACKGROUND

Protein biosynthesis and degradation is a dynamic process which sustains normal cell homeostasis. The ubiquitin-proteasome system is a master regulator of protein homeostasis, by which proteins are initially targeted for poly-ubiquitination by E3 ligases and then degraded into short peptides by the proteasome. Nature evolved diverse peptidic motifs, termed degrons, to signal substrates for degradation. A need exists for the development of methods that efficiently and accurately assess the structural basis of E3 ligase degron recognition and identify proteins capable of being targeted for degradation by the E3 ligase machinery, for example, in the presence of an E3 ligase binding modulator.

### SUMMARY

Cereblon (CRBN) forms an E3 ubiquitin ligase complex with damaged DNA binding protein 1 (DDB1), Cullin-4A (CUL4A), and regulator of cullins 1 (ROC1). This complex ubiquitinates a number of other proteins and can be manipulated with E3 ligase binding modulators such as targeted protein degraders, e.g., small molecules, to trigger targeted degradation of specific substrate proteins of interest. In some cases, binding of substrate proteins with the E3 ubiquitin ligase complex occurs if certain features, known as degrons (e.g., G-loop degrons), are present on the substrate proteins.

In some cases, small molecules modulate the substrate selectivity of CBRN-containing E3 ligases. A need exists for alternative methods for the identification of candidate substrate proteins of the E3 ligase machinery. Described herein, among other things, are computational methods for the identification of candidate substrate proteins of the E3 ligase machinery.

Described herein are methods of identifying a candidate substrate protein for cereblon, the method comprising: identifying a test protein comprising a test amino acid motif having the following formula: X¹—X²—X³—X⁴—X⁵—Y; wherein: Y is 1 to 10 amino acids of the formula X⁶, X⁶—X⁷, X⁶—X⁷—X⁸, X⁶—X⁷—X⁸—X⁹, X⁶—X⁷—X⁸—X⁹—X¹⁰, X⁶—X⁷—X⁸—X⁹—X¹⁰—X¹¹, X⁶—X⁷—X⁸—X⁹—X¹⁰—X¹¹—X¹², X⁶—X⁷—X⁸—X⁹—X¹⁰—X¹¹—X¹²—X¹³, X⁶—X⁷—X⁸—X⁹—X¹⁰—X¹¹—X¹²—X¹³—X¹⁴, or X⁶—X⁷—X⁸—X⁹—X¹⁰—X¹¹—X¹²—X¹³—X¹⁴—X¹⁵, wherein each X is a single amino acid, and wherein X⁵ is glycine, while each of the remaining amino acids are independently selected from any one of the natural occurring amino acids; identifying a corresponding reference amino acid motif from the protein sequence of a known substrate protein for cereblon, wherein the reference amino acid motif is of the same length in amino acids as the test amino acid motif, and wherein the reference amino acid motif has a glycine at amino acid position 5 within the motif; providing a three-dimensional structure for each of the test amino acid motif and the reference amino acid motif; comparing the three-dimensional structure of the test protein's amino acid motif and the reference amino acid motif; based on the comparison, classifying the test protein as a candidate substrate protein for cereblon or not; and optionally: determining one or more additional three-dimensional characterization score(s); and, based on the one or more additional three-dimensional characterization score(s), re-classifying the test protein as a candidate substrate protein for cereblon or not.

In some embodiments, the method further comprises: testing the candidate substrate protein in an E3 ligase substrate detection assay or having the candidate substrate protein tested in an E3 ligase substrate detection assay.

In some embodiments, comparing the three-dimensional structure of the test protein's amino acid motif and the reference amino acid motif comprises: (i) providing the three-dimensional coordinates of the Cα atoms for each amino acid in the test protein amino acid motif and for each amino acid in the reference amino acid motif; (ii) calculating the Binet-Cauchy fragment similarity score (bc-score) between the test protein amino acid motif and the reference amino acid motif.

In some embodiments, the test protein is classified as a candidate substrate protein for cereblon if the be-score is above 0.5, 0.55, 0.6, 0.65, 0.7, 0.75, 0.8, or 0.85.

In some embodiments, the known substrate protein for cereblon is selected from the group consisting of ZNF692, GSPT1, CK1alpha, IKZF1, ZNF692, and SALL4.

In some embodiments, providing the three-dimensional structure for the reference amino acid motif comprises providing a crystal structure selected from the group consisting of ZNF692 PDB 6H0G, GSPT1 PDB 5HXB, GSPT1 PDB 6XK6, CK1alpha PDB 5FQD, IKZF1 PDB 6H0F, ZNF692 PDB 6H0G, SALL4 PDB 6UML, SALL4 PDB 7BQV, or SALL4 PDB 7BQU.

In some embodiments, providing the three-dimensional structure for the reference amino acid motif comprises providing an AlphaFold2 structure selected from the group consisting of “Zinc finger protein 692, Q9BU19 (ZN692_HUMAN)”, “DNA-binding protein IKaros, Q13422 (IKZF1_HUMAN)”, “Sal-like protein 4, Q9UJQ4 (SALL4_HUMAN)”, “Casein kinase I isoform alpha, P48729, (KC1A_HUMAN)”, and “Eukaryotic peptide chain release factor GTP-binding subunit ERF3A, P15170, ERF3A_HUMAN”.

In some embodiments, the reference protein is ZNF692 and the reference amino acid motif begins at position 419 of SEQ ID NO: 8 and comprises 6, 7, 8, 9, 10, 11, 12, 13, 14, or 15 consecutive amino acids of SEQ ID NO: 8 (oriented N- to C- terminally from the beginning position), wherein the number of amino acids in the reference amino acid motif is the same as the number of amino acids in the test protein motif.

In some embodiments, the reference protein is IKZF1 and the reference amino acid motif begins at position 147 of SEQ ID NO: 9 and comprises 6, 7, 8, 9, 10, 11, 12, 13, 14, or 15 consecutive amino acids of SEQ ID NO: 9 (oriented N- to C- terminally from the beginning position), wherein the number of amino acids in the reference amino acid motif is the same as the number of amino acids in the test protein motif.

In some embodiments, the reference protein is SALL4 and the reference amino acid motif begins at position 412 of SEQ ID NO: 10 and comprises 6, 7, 8, 9, 10, 11, 12, 13, 14, or 15 consecutive amino acids of SEQ ID NO: 10 (oriented N- to C- terminally from the beginning position), wherein the number of amino acids in the reference amino acid motif is the same as the number of amino acids in the test protein motif.

In some embodiments, the reference protein is CK1alpha and the reference amino acid motif begins at position 36 of SEQ ID NO: 11 and comprises 6, 7, 8, 9, 10, 11, 12, 13, 14, or 15 consecutive amino acids of SEQ ID NO: 11 (oriented N- to C- terminally from the beginning position), wherein the number of amino acids in the reference amino acid motif is the same as the number of amino acids in the test protein motif.

In some embodiments, the reference protein is GSPT1 and the reference amino acid motif begins at position 433 of SEQ ID NO: 12 and comprises 6, 7, 8, 9, 10, 11, 12, 13, 14, or 15 consecutive amino acids of SEQ ID NO: 12 (oriented N- to C- terminally from the beginning position), wherein the number of amino acids in the reference amino acid motif is the same as the number of amino acids in the test protein motif.

In some embodiments, providing the three dimensional structure for the test protein comprises providing a crystal structure.

In some embodiments, providing the three dimensional structure for the test protein comprises providing a computer modelled three-dimensional structure.

In some embodiments, Y consists of X⁶.

In some embodiments, Y consists of X⁶—X⁷.

In some embodiments, the amino acid motif is at least 8 amino acids long.

In some embodiments, X¹ is aspartic acid (D) or asparagine (N); and wherein X⁴ is serine (S) or threonine (T).

In some embodiments, X¹ and X⁴ are the same.

In some embodiments, X¹ and X⁴ are both cysteine (C); or wherein X¹ and X⁴ are both asparagine (N).

In some embodiments, the E3 ligase substrate detection assay is carried out in the presence of an E3 ligase binding modulator.

In some embodiments, determining one or more additional three-dimensional characterization score(s); and, based on the one or more additional three-dimensional characterization score(s), re-classifying the test protein as a candidate substrate protein for cereblon or not is not optional.

In some embodiments, the E3 ligase binding modulator is a targeted protein degrader.

In some embodiments, the one or more additional three-dimensional characterization score(s) are selected from the group consisting of structural context score(s), atomic distance score(s), cereblon binding compatibility score(s), surface accessibility score(s), geometry score(s), and combinations thereof

Also described herein are uses of predicted degron(s) for identifying a candidate substrate protein for cereblon, wherein the predicted degron is a test amino acid motif within a test protein, wherein the amino acid motif has the following formula: X¹—X²—X³—X⁴—X⁵—Y; wherein: Y is 1 to 10 amino acids of the formula X⁶, X⁶—X⁷, X⁶—X⁷—X⁸, X⁶—X⁷—X⁸—X⁹, X⁶—X⁷—X⁸—X⁹—X¹⁰, X⁶—X⁷—X⁸—X⁹—X¹⁰—X¹¹, X⁶—X⁷—X⁸—X⁹—X¹⁰—X¹¹—X¹², X⁶—X⁷—X⁸—X⁹—X¹⁰—X¹¹—X²—X¹³, X⁶—X⁷—X⁸—X⁹—X¹⁰—X¹¹—X¹²—X¹³—X¹⁴, or X⁶—X⁷—X⁸—X⁹—X¹⁰—X¹¹—X¹²—X¹³—X¹⁴—X¹⁵, wherein each X is a single amino acid, and wherein X⁵ is glycine, while each of the remaining amino acids are independently selected from any one of the natural occurring amino acids; and wherein the use comprises: identifying a corresponding reference amino acid motif from the protein sequence of a known substrate protein for cereblon, wherein the reference amino acid motif is of the same length in amino acids as the test amino acid motif, and wherein the reference amino acid motif has a glycine at amino acid position 5 within the motif; providing a three-dimensional structure for each of the test amino acid motif and the reference amino acid motif; comparing the three-dimensional structure of the test protein's amino acid motif and the reference amino acid motif; based on the comparison, classifying the test protein as a candidate substrate protein for cereblon or not; and optionally: determining one or more additional three-dimensional characterization score(s); and, based on the one or more additional three-dimensional characterization score(s), re-classifying the test protein as a candidate substrate protein for cereblon or not.

In some embodiments, the use further comprises: testing the candidate substrate protein in an E3 ligase substrate detection assay or having the candidate substrate protein tested in an E3 ligase substrate detection assay.

In some embodiments, comparing the three-dimensional structure of the test protein's amino acid motif and the reference amino acid motif comprises: (i) providing the three-dimensional coordinates of the Cα atoms for each amino acid in the test protein amino acid motif and for each amino acid in the reference amino acid motif; (ii) calculating the Binet-Cauchy fragment similarity score (bc-score) between the test protein amino acid motif and the reference amino acid motif.

In some embodiments, the test protein is classified as a candidate substrate protein for cereblon if the be-score is above 0.5, 0.55, 0.6, 0.65, 0.7, 0.75, 0.8, or 0.85.

In some embodiments, the known substrate protein for cereblon is selected from the group consisting of ZNF692, GSPT1, CK1alpha, IKZF1, ZNF692, and SALL4.

In some embodiments, providing the three-dimensional structure for the reference amino acid motif comprises providing a crystal structure selected from the group consisting of ZNF692 PDB 6H0G, GSPT1 PDB 5HXB, GSPT1 PDB 6XK6, CK1alpha PDB 5FQD, IKZF1 PDB 6H0F, ZNF692 PDB 6H0G, SALL4 PDB 6UML, SALL4 PDB 7BQV, or SALL4 PDB 7BQU.

In some embodiments, providing the three-dimensional structure for the reference amino acid motif comprises providing an AlphaFold2 structure selected from the group consisting of “Zinc finger protein 692, Q9BU19 (ZN692_HUMAN)”, “DNA-binding protein IKaros, Q13422 (IKZF1_HUMAN)”, “Sal-like protein 4, Q9UJQ4 (SALL4_HUMAN)”, “Casein kinase I isoform alpha, P48729, (KC1A_HUMAN)”, and “Eukaryotic peptide chain release factor GTP-binding subunit ERF3A, P15170, ERF3A_HUMAN”.

In some embodiments, the reference protein is ZNF692 and the reference amino acid motif begins at position 419 of SEQ ID NO: 8 and comprises 6, 7, 8, 9, 10, 11, 12, 13, 14, or 15 consecutive amino acids of SEQ ID NO: 8 (oriented N- to C- terminally from the beginning position), wherein the number of amino acids in the reference amino acid motif is the same as the number of amino acids in the test protein motif.

In some embodiments, the reference protein is IKZF1 and the reference amino acid motif begins at position 147 of SEQ ID NO: 9 and comprises 6, 7, 8, 9, 10, 11, 12, 13, 14, or 15 consecutive amino acids of SEQ ID NO: 9 (oriented N- to C- terminally from the beginning position), wherein the number of amino acids in the reference amino acid motif is the same as the number of amino acids in the test protein motif.

In some embodiments, the reference protein is SALL4 and the reference amino acid motif begins at position 412 of SEQ ID NO: 10 and comprises 6, 7, 8, 9, 10, 11, 12, 13, 14, or 15 consecutive amino acids of SEQ ID NO: 10 (oriented N- to C- terminally from the beginning position), wherein the number of amino acids in the reference amino acid motif is the same as the number of amino acids in the test protein motif.

In some embodiments, the reference protein is CK1alpha and the reference amino acid motif begins at position 36 of SEQ ID NO: 11 and comprises 6, 7, 8, 9, 10, 11, 12, 13, 14, or 15 consecutive amino acids of SEQ ID NO: 11 (oriented N- to C- terminally from the beginning position), wherein the number of amino acids in the reference amino acid motif is the same as the number of amino acids in the test protein motif.

In some embodiments, the reference protein is GSPT1 and the reference amino acid motif begins at position 433 of SEQ ID NO: 12 and comprises 6, 7, 8, 9, 10, 11, 12, 13, 14, or 15 consecutive amino acids of SEQ ID NO: 12 (oriented N- to C- terminally from the beginning position), wherein the number of amino acids in the reference amino acid motif is the same as the number of amino acids in the test protein motif.

In some embodiments, providing the three dimensional structure for the test protein comprises providing a crystal structure.

In some embodiments, providing the three dimensional structure for the test protein comprises providing a computer modelled three-dimensional structure.

In some embodiments, Y consists of X⁶.

In some embodiments, Y consists of X⁶—X⁷.

In some embodiments, the amino acid motif is at least 8 amino acids long.

In some embodiments, X¹ is aspartic acid (D) or asparagine (N); and wherein X⁴ is serine (S) or threonine (T).

In some embodiments, X¹ and X⁴ are the same.

In some embodiments, X¹ and X⁴ are both cysteine (C); or wherein X¹ and X⁴ are both asparagine (N).

In some embodiments, the E3 ligase substrate detection assay is carried out in the presence of an E3 ligase binding modulator.

In some embodiments, determining one or more additional three-dimensional characterization score(s); and, based on the one or more additional three-dimensional characterization score(s), re-classifying the test protein as a candidate substrate protein for cereblon or not is not optional.

In some embodiments, the E3 ligase binding modulator is a targeted protein degrader.

In some embodiments, the one or more additional three-dimensional characterization score(s) are selected from the group consisting of structural context score(s), atomic distance score(s), cereblon binding compatibility score(s), surface accessibility score(s), geometry score(s), and combinations thereof

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1. depicts a non-limiting discovery process for an in silico analysis and cataloguing of G-loop containing proteins.

FIG. 2. shows G-loop degron matches for human catalase. G-loop matches found with a BC score>0.89 using a 5 aa [‘. . . G’] probe. None of these are found with a 6 aa-length search probe (top BC score=0.86 also FP).

### DETAILED DESCRIPTION

The ubiquitin-proteasome system (UPS) is a complex cellular pathway by which proteins are first ubiquitinated and subsequently unfolded and proteolyzed by the proteasome. This process has direct implications primarily on regulating protein homeostasis and, depending on the context, can impact many cellular signaling processes, including, but not limited to, DNA repair, apoptosis, inflammation, transcription regulation, stress response, and protein quality control. Three main classes of enzymes are responsible for the specific targeting of proteins for degradation: E1-activating enzymes, which activate ubiquitin (Ub) in an ATP-dependent manner; E2-conjugating enzymes, to which the activated Ub is covalently attached to yield an E2˜Ub thioester intermediate; and E3 ubiquitin ligases, which catalyze the transfer of Ub from the E2 enzyme to form an isopeptide bond with a lysine residue on the protein substrate (mono-ubiquitination or priming) or its covalently attached Ub (poly-ubiquitination). To act as catalyst in the process, E3 ligases typically recruit specific target substrates for degradation by recognition of peptidic segments termed ‘degrons’. The structural features of the degron and its cognate E3 ubiquitin ligase confer substrate specificity and determine protein recognition and fate are important to elucidate and be able to manipulate proteasome-mediated degradation.

The large number of E3 ligase proteins (>600) encoded in the human genome and the diversity and specificity of degron motifs provide numerous opportunities for drug development. To date, only a handful of E3 ligases (including CRBN, VHL, IAP and MDM2) have been effectively hijacked by small-molecules.

The ubiquitin proteasome system can be manipulated with different small molecules to trigger targeted degradation of specific proteins of interest. Promoting the targeted degradation of disease-relevant proteins using small molecule degraders is emerging as a new modality in the treatment of diseases. One such modality relies on redirecting the activity of E3 ligases such as cereblon (a phenomenon known as E3 reprogramming) using small molecule binders, which have been termed molecular glue degraders (Tan et al. Nature 2007, 446, 640-645 and Sheard et al. Nature 2010, 468, 400-405) to promote the poly-ubiquitination and ultimately proteasomal degradation of new protein substrates involved in the development of diseases. The molecular glues bind to both the E3 ligase and the target protein, thereby mediating an alteration of the ligase surface and enabling an interaction with the target protein. Particular relevant compounds for the E3 ligase cereblon are the IMiD (immunomodulatory imide drugs) class including Thalidomide, Lenalidomide and Pomalidomide. These IMiDs have been approved by the FDA for use in hematological cancers. However, compounds for efficiently targeting other diseases and proteins, that would benefit therapeutically from the degradation, e.g., the targeted degradation, of a protein(s), in particular other types of cancers, and technologies and methods for designing, e.g., rationally designing, such compounds, are still required.

The disclosure herein provides such technologies and methods. Specifically, the compositions and methods described herein are useful, for example, in identification and/or prediction of proteins that contain one or more degrons.

Degrons are structural features of proteins that facilitate recruitment to and subsequent degradation by an E3 ligase complex, e.g., an E3 ligase complex described herein. Degrons are described, for example, in Lucas and Ciulli, “Recognition of Substrate Dependent Degrons by E3 Ubiquitin Ligases and Modulation by Small-Molecule Mimicry Strategies,” Current Opinion in Structural Biology 44:101-10 (2017).

In some cases, the degron is a small molecule dependent degron (i.e., is a structural feature on the surface of the protein that mediates recruitment of and degradation by an E3 ligase only in the presence of a targeted protein degrader). In some cases, the degron is a small molecule independent degron (i.e., is a structural feature on the surface of the protein that mediates recruitment of and degradation by an E3 ligase in the absence of a targeted protein degrader). Proteins containing small molecule dependent degrons are sometimes referred to as “neosubstrates,” whereas proteins containing small molecule independent degrons are sometimes referred to as “substrates.” Unless otherwise indicated, a “candidate cereblon substrate,” as used herein, encompasses proteins comprising either or both of small molecule dependent and small molecule independent degron(s).

Degrons include, e.g., G-loop degrons. Thus, in some cases, the E3 ligase binding target is a protein comprising an E3 ligase-accessible loop, e.g., a cereblon-accessible loop, e.g., a G-loop.

Thus, described herein are, among other things, methods for identifying candidate substrate proteins for cereblon.

### Cereblon

The cereblon protein, encoded by the gene CRBN, is the substrate recognition component of a DCX (DDB1-CUL4—X-box) E3 protein ligase complex that mediates the ubiquitination and subsequent proteasomal degradation of target proteins.

The human cereblon protein (NCBI Gene ID 51185; UniProt ID Q96SW2) encodes the following transcripts and isoforms, of which NM_016302.4 (SEQ ID NO: 2, transcript 1) is the canonical transcript:

| | Length | | Length | | |
|---|---|---|---|---|---|
| Transcript | (nt) | Protein | (aa) | SEQ ID NO: | Isoform |
| XR_940448.3 | 2667 | | | | |
| XM_011533791.3 | 3586 | XP_011532093.1 | 398 | SEQ ID NO: 4 | X1 |
| XM_011533793.2 | 2927 | XP_011532095.1 | 278 | SEQ ID NO: 5 | X4 |
| XM_011533794.2 | 2798 | XP_011532096.1 | 278 | SEQ ID NO: 6 | X4 |
| NM_001173482.1 | 2593 | NP_001166953.1 | 441 | SEQ ID NO: 1 | 2 |
| XM_005265202.4 | 2472 | XP_005265259.1 | 379 | SEQ ID NO: 3 | X2 |
| NM_016302.4 | 2187 | NP_057386.2 | 442 | SEQ ID NO: 2 | 1 |
| XM_024453551.1 | 1458 | XP_024309319.1 | 284 | SEQ ID NO: 7 | X3 |

Isoform 1 of human CRBN (SEQ ID NO: 2) has the following features:

| Feature | Position(s) | Reference |
|---|---|---|
| Zinc binding | 323 | Chamberlain et al. Nat. Struct. Mol. |
| Zinc binding | 326 | Biol. 21: 803-9 (2014) |
| Zinc binding | 391 | |
| Zinc binding | 394 | |

Known mutants of human CRBN isoform 1 (SEQ ID NO: 2) have the following features:

| Feature key | Position(s) | Description | Reference(s) |
|---|---|---|---|
| Mutagenesis | 384 | Y → A: Abolishes | Ito et al., Science 327: 1345-50 |
| | | thalidomide-binding without | (2010) |
| | | affecting DCX protein ligase | |
| | | complex activity; when | |
| | | associated with A-386. | |
| Mutagenesis | 386 | W → A: Abolishes | Ito et al., Science 327: 1345-50 |
| | | thalidomide-binding without | (2010); |
| | | affecting DCX protein ligase | Chamberlain et al. Nat. Struct. |
| | | complex activity; when | Mol. Biol. 21: 803-9 (2014) |
| | | associated with A-384. | |
| | | Abolishes pomalidomide- | |
| | | induced change in substrate | |
| | | specificity and abolishes | |
| | | pomalidomide-induced | |
| | | decrease in cell viability that is | |
| | | brought about by increased | |
| | | degradation of MYC, IRF4 | |
| | | and IKZF3. | |
| Mutagenesis | 419-442 | Missing: Fails to rescue | Choi et al., J. Neurosci. |
| | | increased BK channel activity | 38: 3571-83 (2018) |
| | | and decreased probability of | |
| | | neurotransmission in a mouse | |
| | | hippocampal neuron model. | |

Isoform 1 of human CRBN (SEQ ID NO: 2) comprises a Lon N-terminal domain at positions 81-317, the canonical binding domain CULT (cereblon domain of unknown activity, binding cellular Ligands and; Thalomide) at positions 318-426, and canonical thalomide binding region at positions 378-386 (Chamberlain et al. Nat. Struct. Mol. Biol. 21:803-9 (2014)). The CULT domain binds thalidomide and related drugs, such as pomalidomide and lenalidomide. Drug binding leads to a change in substrate specificity of the human DCX (DDB1-CUL4—X-box) E3 protein ligase complex, while no such change is observed in rodents (Chamberlain et al. Nat. Struct. Mol. Biol. 21:803-9 (2014)).

In some cases, the cereblon protein is human cereblon protein. In some cases, the cereblon protein comprises or consists of SEQ ID NO: 1, SEQ ID NO: 2, SEQ ID NO: 3, SEQ ID NO: 4, SEQ ID NO: 5, SEQ ID NO: 6, or SEQ ID NO: 7. In some cases, the cerebelon protein is at least 80% identical to SEQ ID NO: 1, SEQ ID NO: 2, SEQ ID NO: 3, SEQ ID NO: 4, SEQ ID NO: 5, SEQ ID NO: 6, or SEQ ID NO: 7, e.g., at least 90%, at least 95% or at least 99% identical to SEQ ID NO: 1, SEQ ID NO: 2, SEQ ID NO: 3, SEQ ID NO: 4, SEQ ID NO: 5, SEQ ID NO: 6, or SEQ ID NO: 7.

The cereblon protein comprises a central LON domain (residues 80-317) followed by a C-terminal CULT domain. The LON domain is further subdivided into an N-terminal LON-N subdomain, a four α-helix bundle, and a C-terminal LON-C subdomain. In humans, the cereblon gene has been identified as a candidate gene of an autosomal recessive nonsyndromic mental retardation (ARNSMR) (Higgins, J. J. et al, Neurology, 2004, 63: 1927-193).

Cereblon was initially characterized as an RGS-containing novel protein that interacted with a calcium-activated potassium channel protein (SLO1) in the rat brain, and was later shown to interact with a voltage-gated chloride channel (CIC-2) in the retina with AMPK1 and DDB1. See Jo, S. et al, J. Neurochem, 2005, 94: 1212-1224; Hohberger B. et al, FEBS Lett, 2009, 583: 633-637; Angers S. et al, Nature, 2006, 443: 590-593. DDB1 was originally identified as a nucleotide excision repair protein that associates with damaged DNA binding protein 2 (DDB2). Its defective activity causes the repair defect in the patients with xeroderma pigmentosum complementation group E (XPE). DDB1 also appears to function as a component of numerous distinct DCX (DDB1-CUL4—X-box) E3 ubiquitin-protein ligase complexes which mediate the ubiquitination and subsequent proteasomal degradation of target proteins.

Binding of small molecules to CBRN have been shown to induce recruitment and degradation of protein substrates such as, but not limited to, GSPT1. These observations demonstrate that substrate selectivity of E3 ligases can be effectively modulated by binding of small molecules, which can act either as stabilizers or disruptors of specific E3 ligase:degron complexes. As expected, a need exists for the identification of protein substrates for E3 ligases (including CRBN) that have been effectively hijacked by small-molecules, whose structures are known or yet to be identified.

### Amino Acid Motifs

In some cases, the methods described herein comprise identifying a test protein that comprises a particular amino acid motif. In some cases the amino acid motif is X¹—X²—X³—X⁴—X⁵—Y; wherein: Y is 1 to 10 amino acids of the formula X⁶, X⁶—X⁷, X⁶—X⁷—X⁸, X⁶—X⁷—X⁸—X⁹, X⁶—X⁷—X⁸—X⁹—X¹⁰, X⁶—X⁷—X⁸—X⁹—X¹⁰—X¹¹, X⁶—X⁷—X⁸—X⁹—X¹⁰—X¹¹—X¹², X⁶—X⁷—X⁸—X⁹—X¹⁰—X¹¹—X¹²—X¹³, X⁶—X⁷—X⁸—X⁹—X¹⁰—X¹¹—X¹²—X¹³—X¹⁴, or X⁶—X⁷—X⁸—X⁹—X¹⁰—X¹¹—X¹²—X¹³—X¹⁴—X¹⁵, and wherein X⁵ is glycine, while each of the remaining amino acids (X^{N}) are independently selected from any one of the natural occurring amino acids.

In some embodiments, the method comprises searching a database, e.g., a protein database, for a protein comprising the amino acid motif. In some embodiments, the database includes a protein data bank (PDB) database.

In some embodiments, searching comprises searching a protein database for a protein comprising a specific amino acid sequence motif that has between 5 to about 15 amino acids. In some embodiments, the amino acid sequence motif comprise between 6 to 14, 6 to 13, 6 to 12, 6 to 11, 6 to 10, 6 to 9, or 6 to 8 amino acids. In some embodiments, the amino acid sequence motif comprises 6, 7, 8, 9, 10, 11, 12, 13, 14, or 15 amino acids. In some embodiments, the amino acid sequence motif comprises 6 amino acids. In some embodiments, the amino acid sequence motif comprises 7 amino acids. In some embodiments, the amino acid sequence motif comprises 8 amino acids. In some embodiments, the amino acid sequence motif comprises 9 amino acids. In some embodiments, the amino acid sequence motif comprises 10 amino acids.

In some embodiments, the amino acid motif is 6 amino acids having the following formula: X¹—X²—X³—X⁴—X⁵—X⁶; wherein: each of X¹, X², X³, X⁴, and X⁶ are independently selected from any one of the natural occurring amino acids; and X⁵ is G (i.e. glycine).

In some embodiments, the amino acid motif is 7 amino acids having the following formula: X¹—X²—X³—X⁴—X⁵—X⁶—X⁷; wherein: each of X¹, X², X³, X⁴, X⁶, and X⁷ are independently selected from any one of the natural occurring amino acids; and X⁵ is G (i.e. glycine).

In some embodiments, the amino acid motif is 8 amino acids having the following formula: X¹—X²—X³—X⁴—X⁵—X⁶—X⁷—X⁸; wherein: each of X¹, X², X³, X⁴, X⁶, X⁷, and X⁸ are independently selected from any one of the natural occurring amino acids; and X⁵ is G (i.e. glycine).

In some embodiments, the amino acid motif is 9 amino acids having the following formula: X¹—X²—X³—X⁴—X⁵—X⁶—X⁷—X⁸—X⁹; wherein: each of X¹, X², X³, X⁴, X⁶, X⁷, X⁸, and X⁹ are independently selected from any one of the natural occurring amino acids; and X⁵ is G (i.e. glycine).

In some embodiments, the amino acid motif is 10 amino acids having the following formula: X¹—X²—X³—X⁴—X⁵—X⁶—X⁷—X⁸—X⁹—X¹⁰; wherein: each of X¹, X², X³, X⁴, X⁶, X⁷, X⁸, X⁹, and X¹⁰ are independently selected from any one of the natural occurring amino acids; and X⁵ is G (i.e. glycine).

In some embodiments, the amino acid motif is 11 amino acids having the following formula: X¹—X²—X³—X⁴—X⁵—X⁶—X⁷—X⁸—X⁹—X¹⁰—X¹¹; wherein: each of X¹, X², X³, X⁴, X⁶, X⁷, X⁸, X⁹, X¹⁰, and X¹¹ are independently selected from any one of the natural occurring amino acids; and X⁵ is G (i.e. glycine).

In some embodiments, the amino acid motif is 12 amino acids having the following formula: X¹—X²—X³—X⁴—X⁵—X⁶—X⁷—X⁸—X⁹—X¹⁰—X¹¹—X¹²; wherein: each of X¹, X², X³, X⁴, X⁶, X⁷, X⁸, X⁹, X¹⁰, X¹¹, and X¹² are independently selected from any one of the natural occurring amino acids; and X⁵ is G (i.e. glycine).

In some embodiments, the amino acid motif is 13 amino acids having the following formula: X¹—X²—X³—X⁴—X⁵—X⁶—X⁷—X⁸—X⁹—X¹⁰—X¹¹—X¹²—X¹³; wherein: each of X¹, X², X³, X⁴, X⁶, X⁷, X⁸, X⁹, X¹⁰, X¹¹, X¹², and X¹³ are independently selected from any one of the natural occurring amino acids; and X⁵ is G (i.e. glycine).

In some embodiments, the amino acid motif is 14 amino acids having the following formula: X¹—X²—X³—X⁴—X⁵—X⁶—X⁷—X⁸—X⁹—X¹⁰—X¹¹—X¹²—X¹³—X¹⁴; wherein: each of X¹, X², X³, X⁴, X⁶, X⁷, X⁸, X⁹, X¹⁰, X¹¹, X¹², X¹³, and X¹⁴ are independently selected from any one of the natural occurring amino acids; and X⁵ is G (i.e. glycine).

In some embodiments, amino acids at positions X¹ and X⁴ are the same. In some embodiments, amino acids at positions X¹ and X⁴ are different amino acids.

In some embodiments, X¹ is aspartic acid or asparagine and X⁴ is serine or threonine.

In some embodiments, the amino acid motif is represented by the formula [D/N]X² X^{3 [}S/T]GX⁶, wherein position X¹ is aspartic acid or asparagine, X⁴ is serine or threonine, and X², X³ and X⁶ are any one of the naturally occurring amino acids.

In some embodiments, the degron comprises a 6 amino acid motif represented by formula [D/N]X² X^{3 [}S/T]GX⁶. In some embodiments, the degron comprises a motif represented by formula CX² X³ CGX⁶ or NX² X³ NGX⁶. In some embodiments, the degron comprises a 7 amino acid motif represented by formula [D/N]X² X^{3 [}S/T]GX⁶ X⁷. In some embodiments, the degron comprises a motif represented by formula CX² X³ CGX⁶ X⁷ or NX² X³ NGX⁶ X⁷. In some embodiments, the degron comprises a 8 amino acid motif represented by formula [D/N]X² X^{3 [}S/T]GX⁶ X⁷ X⁸. In some embodiments, the degron comprises a motif represented by formula CX² X³ CGX⁶ X⁷ X⁸ or NX² X³ NGX⁶ X⁷X⁸.

The methods provided herein provide an improvement over methods that utilize a degron comprising an amino acid sequence of 5 or fewer amino acids. Increasing the amino acid chain length searched from 5 to at least 6 amino acids reduces the number of false positive hits. For example, a search for proteins with a motif length of 5 amino acids or less results in the identification of amino acid sequences present in helices of the protein, in addition to loop(s). Increasing the amino acid sequence to at least 6 amino acids reduces or eliminates the identification of the amino acid sequences in portions of proteins other than G-loop(s).

These motifs, when found in a test protein, are also referred to herein as “predicted degrons”.

Three-dimensional Structures

In some cases, the methods described herein include providing a three-dimensional structure. In some cases, the three-dimensional structure is a crystal structure. In some cases, the crystal structure is ligand bound (i.e. holo). In some cases, the crystal structure is unbound (i.e. apo).

In some cases, the three-dimensional structure is obtained from a database. For example, the Protein Data Bank (PDB) or the AlphaFold Protein Structure Database (alphafold.ebi.ac.uk).

PDB is a database for the three-dimensional structural data of large biological molecules, such as proteins and nucleic acids (Nucleic Acids Res. 2019 Jan 8;47(D1):D520-D528. doi: 10.1093/nar/gky949). The data is submitted by biologists and biochemists from around the world, are freely accessible on the Internet via the websites of its member organizations (e.g. PDBe—pdbe.org, PDBj—pdbj.org, RCSB—rcsb.orgipdb, and BMRB—bmrb.wisc.edu). The PDB is overseen by an organization called the Worldwide Protein Data Bank—wwPDB-.

In some embodiments, providing a three-dimensional structure comprises generating a three-dimensional structure, e.g., crystal structure.

In some embodiments, providing a three-dimensional structure comprises computer modeling of the three-dimensional structural context, e.g., if the three-dimensional structure of the identified protein is not known. In some cases, computer modeling of the three-dimensional structural context is carried out using an artificial intelligence program, e.g., according to the methods described in Jumper et al., “Highly Accurate Protein Structure Prediction with AlphaFold,” Nature 596:583-89 (2021) or Evans et al., “Protein Complex Prediction with AlphaFold-Multimer,” bioRxiv doi.org/10.1101/2021.10.04.463034 (2021).

In some cases, the three-dimensional structure of the test protein is a homologue of the candidate cereblon target. For example, where the candidate cereblon target is a human protein, a three-dimensional structure of a homologous non-human animal protein may be used as the three-dimensional structure of the candidate cereblon target. This is useful, for example, where there is a crystal structure available for a homologous protein but not the candidate cereblon target itself.

### Reference Proteins

In some cases, the methods described herein include reference protein(s), e.g., known substrate protein(s) for cereblon. In some cases, the reference protein is a known substrate protein for cereblon in the absence of an E3 ligase binding modulator. In some cases, the reference protein is a known substrate protein for cereblon in the presence of an E3 ligase binding modulator.

In some cases, the methods include identifying a corresponding reference motif in a known substrate for cereblon. In some cases, the corresponding reference motif is a portion of the protein sequence for a known substrate for cereblon. In some cases, the corresponding reference motif is the same length in amino acids as the test protein amino acid motif. In some cases, the corresponding reference motif has a glycine at position 5 within the motif (oriented N- to C- terminally from the beginning position of the motif).

In some cases, the known substrate for cereblon is selected from the group consisting of ZNF692, GSPT1, CK1alpha, IKZF 1, and SALL4.

### ZNF692

In some cases, the reference protein is ZNF692 (UniProt ID Q9BU19; SEQ ID NO: 8, shown below).

>sp|Q9BU19|ZN692_HUMAN Zinc finger protein 692
OS = Homo sapiens OX = 9606 GN = ZNF692 PE = 1
SV = 1
MASSPAVDVSCRRREKRRQLDARRSKCRIRLGGHMEQWCLLKERLGFSLH
SQLAKELLDRYTSSGCVLCAGPEPLPPKGLQYLVLLSHAHSRECSLVPGL
RGPGGQDGGLVWECSAGHTFSWGPSLSPTPSEAPKPASLPHTTRRSWCSE
ATSGQELADLESEHDERTQEARLPRRVGPPPETFPPPGEEEGEEEEDNDE
DEEEMLSDASLWTYSSSPDDSEPDAPRLLPSPVTCTPKEGETPPAPAALS
SPLAVPALSASSLSSRAPPPAEVRVQPQLSRTPQAAQQTEALASTGSQAQ
SAPTPAWDEDTAQIGPKRIRKAAKRELMPCDFPGCGRIFSNRQYLNHHKK
YQHIHQKSESCPEPACGKSENFKKHLKEHMKLHSDTRDYICEFCARSERT
SSNLVIHRRIHTGEKPLQCEICGFTCRQKASLNWHQRKHAETVAALRFPC
EFCGKRFEKPDSVAAHRSKSHPALLLAPQESPSGPLEPCPSISAPGPLGS
SEGSRPSASPQAPTLLPQQ

In some cases, the reference protein is ZNF692 and the reference amino acid motif begins at position 419 of SEQ ID NO: 8 and comprises 6, 7, 8, 9, 10, 11, 12, 13, 14, or 15 consecutive amino acids of SEQ ID NO: 8 (oriented N- to C- terminally from the beginning position). The first six amino acids, beginning at positions 419, are bolded and underlined in SEQ ID NO: 8, above.

In some cases, the methods described herein include providing a three-dimensional structure of a reference protein. In some cases, the reference protein is ZNF692 and the three-dimensional structure is PDB entry ZNF692 PDB 6H0G. In some cases, the reference protein is ZNF692 and the three-dimensional structure is AlphaFold Protein Structure Database entry “Zinc finger protein 692, Q9BU19 (ZN692_HUMAN)”.

The coordinates of the Cα atoms of the reference amino acid motif beginning at position 419 of SEQ ID NO: 8, in ZNF692 PDB 6H0G chain C 419-433 aa, are in Table A,

TABLE A
Coordinates for ZNF692

| Motif Position | Amino Acid | X | Y | Z |
|---|---|---|---|---|
| X1 | C | 0.351 | 48.712 | −91.64 |
| X2 | E | −2.483 | 48.832 | −89.184 |
| X3 | I | −1.39 | 45.65 | −87.396 |
| X4 | C | 2.1 | 46.67 | −86.362 |
| X5 | G | 2.773 | 50.141 | −87.785 |
| X6 | F | 5.17 | 48.757 | −90.404 |
| X7 | T | 5.912 | 51.72 | −92.648 |
| X8 | C | 6.6 | 51.112 | −96.411 |
| X9 | R | 6.573 | 52.811 | −99.809 |
| X10 | Q | 5.228 | 51.092 | −102.965 |
| X11 | K | 1.609 | 49.91 | −102.876 |
| X12 | A | 1.741 | 46.13 | −102.941 |
| X13 | S | 4.812 | 45.865 | −100.665 |
| X14 | L | 2.631 | 46.409 | −97.624 |
| X15 | N | 0.104 | 43.996 | −99.185 |

### IKZF1

In some cases, the reference protein is IKZF1 (UniProt ID Q13422; SEQ ID NO: 9, shown below).

>sp|Q13422|IKZF1_HUMAN DNA-binding protein Ikaros
OS = Homo sapiens OX = 9606 GN = IKZF1 PE = 1
SV = 1
MDADEGQDMSQVSGKESPPVSDTPDEGDEPMPIPEDLSTTSGGQQSSKSD
RVVASNVKVETQSDEENGRACEMNGEECAEDLRMLDASGEKMNGSHRDQG
SSALSGVGGIRLPNGKLKCDICGIICIGPNVLMVHKRSHTGERPFQCNQC
GASFTQKGNLLRHIKLHSGEKPFKCHLCNYACRRRDALTGHLRTHSVGKP
HKCGYCGRSYKQRSSLEEHKERCHNYLESMGLPGTLYPVIKEETNHSEMA
EDLCKIGSERSLVLDRLASNVAKRKSSMPQKFLGDKGLSDTPYDSSASYE
KENEMMKSHVMDQAINNAINYLGAESLRPLVQTPPGGSEVVPVISPMYQL
HKPLAEGTPRSNHSAQDSAVENLLLLSKAKLVPSEREASPSNSCQDSTDT
ESNNEEQRSGLIYLTNHIAPHARNGLSLKEEHRAYDLLRAASENSQDALR
VVSTSGEQMKVYKCEHCRVLFLDHVMYTIHMGCHGFRDPFECNMCGYHSQ
DRYEFSSHITRGEHRFHMS

In some cases, the reference protein is IKZF1 and the reference amino acid motif begins at position 147 of SEQ ID NO: 9 and comprises 6, 7, 8, 9, 10, 11, 12, 13, 14, or 15 consecutive amino acids of SEQ ID NO: 9 (oriented N- to C- terminally from the beginning position). The first six amino acids, beginning at positions 147, are bolded and underlined in SEQ ID NO: 9, above.

In some cases, the methods described herein include providing a three-dimensional structure of a reference protein. In some cases, the reference protein is IKZF1 and the three-dimensional structure is PDB entry IKZF1 PDB 6H0F. In some cases, the reference protein is IKZF1 and the three-dimensional structure is AlphaFold Protein Structure Database entry “DNA-binding protein IKaros, Q13422 (IKZF1_HUMAN)”.

The coordinates of the Cα atoms of the reference amino acid motif beginning at position 147 of SEQ ID NO: 9, in IKZF1 PDB entry 6H0F chain B 147-161 aa are as in Table B.

TABLE B
Coordinates for Motif in IKZF1

| Motif Position | Amino Acid | X | Y | Z |
|---|---|---|---|---|
| X1 | C | −34.38 | −65.768 | −16.549 |
| X2 | N | −36.048 | −62.381 | −17.228 |
| X3 | Q | −32.914 | −61.036 | −18.979 |
| X4 | C | −32.346 | −63.769 | −21.631 |
| X5 | G | −35.166 | −66.375 | −21.248 |
| X6 | A | −32.837 | −69.147 | −19.994 |
| X7 | S | −35.051 | −71.858 | −18.404 |
| X8 | F | −33.839 | −74.112 | −15.529 |
| X9 | T | −34.886 | −77.357 | −13.91 |
| X10 | Q | −34.073 | −76.014 | −10.351 |
| X11 | K | −34.317 | −72.479 | −8.835 |
| X12 | G | −30.707 | −72.693 | −7.608 |
| X13 | N | −29.435 | −73.06 | −11.198 |
| X14 | L | −31.475 | −70.011 | −12.241 |
| X15 | L | −30.059 | −68.004 | −9.27 |

### SALL4

In some cases, the reference protein is SALL4 (UniProt ID Q9UJQ4; SEQ ID NO: 10, shown below).

>sp|Q9UJQ4|SALL4_HUMAN Sal-like protein 4
OS = Homo sapiens OX = 9606 GN = SALL4 PE = 1
SV = 1
MSRRKQAKPQHINSEEDQGEQQPQQQTPEFADAAPAAPAAGELGAPVNHP
GNDEVASEDEATVKRLRREETHVCEKCCAEFFSISEFLEHKKNCTKNPPV
LIMNDSEGPVPSEDESGAVLSHQPTSPGSKDCHRENGGSSEDMKEKPDAE
SVVYLKTETALPPTPQDISYLAKGKVANTNVTLQALRGTKVAVNQRSADA
LPAPVPGANSIPWVLEQILCLQQQQLQQIQLTEQIRIQVNMWASHALHSS
GAGADTLKTLGSHMSQQVSAAVALLSQKAGSQGLSLDALKQAKLPHANIP
SATSSLSPGLAPFTLKPDGTRVLPNVMSRLPSALLPQAPGSVLFQSPEST
VALDTSKKGKGKPPNISAVDVKPKDEAALYKHKCKYCSKVFGTDSSLQIH
LRSHTGERPFVCSVCGHRFTTKGNLKVHFHRHPQVKANPQLFAEFQDKVA
AGNGIPYALSVPDPIDEPSLSLDSKPVLVTTSVGLPQNLSSGTNPKDLTG
GSLPGDLQPGPSPESEGGPTLPGVGPNYNSPRAGGFQGSGTPEPGSETLK
LQQLVENIDKATTDPNECLICHRVLSCQSSLKMHYRTHTGERPFQCKICG
REAFSTKGNLKTHLGVHRTNTSIKTQHSCPICQKKFTNAVMLQQHIRMHM
GGQIPNTPLPNPCDFTGSEPMTVGENGSTGAICHDDVIESIDVEEVSSQE
SSAPSSKVPTPLPSIHSASPTLGFAMMASLDAPGKVGPAPFNLQRQGSRE
NGSVESDGLTNDSSSLMGDQEYQSRSPDILETTSFQALSPANSQAESIKS
KSPDAGSKAESSENSRTEMEGRSSLPSTFIRAPPTYVKVEVPGTFVGPST
LSPGMTPLLAAQPRRQAKQHGCTRCGKNESSASALQIHERTHTGEKPFVC
TNICGRAFTKGNLKVHYMTHGANNNSARRGRKLAIENTMALLGTDGKRVS
EIFPKEILAPSVNVDPVVWNQYTSMLNGGLAVKTNEISVIQSGGVPTLPV
SLGATSVVNNATVSKMDGSQSGISADVEKPSATDGVPKHQFPHELEENKI
AVS

In some cases, the reference protein is SALL4 and the reference amino acid motif begins at position 412 of SEQ ID NO: 10 and comprises 6, 7, 8, 9, 10, 11, 12, 13, 14, or 15 consecutive amino acids of SEQ ID NO: 10 (oriented N- to C- terminally from the beginning position). The first six amino acids, beginning at positions 412, are bolded and underlined in SEQ ID NO: 10, above.

In some cases, the methods described herein include providing a three-dimensional structure of a reference protein. In some cases, the reference protein is SALL4 and the three-dimensional structure is PDB entry SALL4 PDB 6UML, SALL4 PDB 7BQV, or SALL4 PDB 7BQU. In some cases, the reference protein is SALL4 and the three-dimensional structure is AlphaFold Protein Structure Database entry “Sal-like protein 4, Q9UJQ4 (SALL4_HUMAN)”.

The coordinates of the Cα atoms of the reference amino acid motif beginning at position 412 of SEQ ID NO: 10, in PDB entry 7BQV chain B 413-426 aa) are in Table C.

TABLE C
Coordinates for Motif in SALL4

| Motif Position | Amino Acid | X | Y | Z |
|---|---|---|---|---|
| X1 | C | −25.038 | −10.682 | −12.021 |
| X2 | S | −23.503 | −14.084 | −12.726 |
| X3 | V | −22.235 | −14.298 | −9.133 |
| X4 | C | −25.387 | −13.69 | −7.05 |
| X5 | G | −28.414 | −13.306 | −9.36 |
| X6 | H | −28.979 | −9.591 | −8.673 |
| X7 | R | −30.974 | −8.139 | −11.592 |
| X8 | F | −30.329 | −4.776 | −13.236 |
| X9 | T | −32.095 | −2.638 | −15.806 |
| X10 | T | −28.86 | −0.991 | −16.987 |
| X11 | K | −25.702 | −2.787 | −18.064 |
| X12 | G | −23.577 | −0.086 | −16.429 |
| X13 | N | −24.915 | −0.751 | −12.94 |
| X14 | L | −24.37 | −4.495 | −13.442 |
| X15 | K | −20.716 | −3.837 | −14.315 |

### CK1alpha

In some cases, the reference protein is CK1alpha (CSNK1A1; UniProt ID P48729; SEQ ID NO: 11, shown below).

>sp|P48729|KC1A_HUMAN Casein kinase I isoform
alpha OS = Homo sapiens OX = 9606 GN = CSNK1A1
PE = 1 SV = 2
MASSSGSKAEFIVGGKYKLVRKIGSGSFGDIYLAINITNGEEVAVKLESQ
KARHPQLLYESKLYKILQGGVGIPHIRWYGQEKDYNVLVMDLLGPSLEDL
ENFCSRRFTMKTVLMLADQMISRIEYVHTKNFIHRDIKPDNFLMGIGRHC
NKLFLIDFGLAKKYRDNRTRQHIPYREDKNLTGTARYASINAHLGIEQSR
RDDMESLGYVLMYFNRTSLPWQGLKAATKKQKYEKISEKKMSTPVEVLCK
GFPAEFAMYLNYCRGLRFEEAPDYMYLRQLFRILFRTLNHQYDYTEDWTM
LKQKAAQQAASSSGQGQQAQTPTGKQTDKTKSNMKGF

In some cases, the reference protein is CK1alpha and the reference amino acid motif begins at position 36 of SEQ ID NO: 11 and comprises 6, 7, 8, 9, 10, 11, 12, 13, 14, or 15 consecutive amino acids of SEQ ID NO: 11 (oriented N- to C- terminally from the beginning position). The first six amino acids, beginning at position 36, are underlined in SEQ ID NO: 11, above.

In some cases, the methods described herein include providing a three-dimensional structure of a reference protein. In some cases, the reference protein is CK1alpha and the three-dimensional structure is PDB entry CK1alpha PDB 5FQD. In some cases, the reference protein is CK1alpha and the three-dimensional structure is AlphaFold2 entry “Casein kinase I isoform alpha, P48729, (KC1A_HUMAN)”.

The coordinates of the Cα atoms of the reference amino acid motif beginning at position 36 of SEQ ID NO: 11, in CK1alpha PDB entry 5FQD chain B 36-50 aa are in Table D.

TABLE D
Coordinate for Motif in CK1alpha

| Motif Position | Amino Acid | X | Y | Z |
|---|---|---|---|---|
| X1 | N | 39.712 | 137.694 | 19.336 |
| X2 | I | 38.425 | 134.078 | 19.156 |
| X3 | T | 38.449 | 134.189 | 15.325 |
| X4 | N | 42.191 | 134.891 | 14.791 |
| X5 | G | 44.229 | 135.187 | 18.077 |
| X6 | E | 44.384 | 139.014 | 18.118 |
| X7 | E | 44.984 | 140.454 | 21.649 |
| X8 | V | 43.008 | 143.526 | 22.694 |
| X9 | A | 42.188 | 145.642 | 25.774 |
| X10 | V | 38.572 | 145.662 | 27.04 |
| X11 | K | 37.451 | 148.466 | 29.423 |
| X12 | L | 34.415 | 147.182 | 31.369 |
| X13 | E | 32.051 | 149.558 | 33.233 |
| X14 | S | 28.937 | 148.623 | 35.119 |
| X15 | Q | 25.693 | 150.103 | 33.779 |

### GSPT1

In some cases, the reference protein is GSPT1 (ERF3A; UniProt ID P15170; SEQ ID NO: 12, shown below).

>sp|P15170|ERF3A_HUMAN Eukaryotic peptide chain
release factorGTP-binding subunit ERF3A OS = Homo
sapiens OX = 9606 GN = GSPT1 PE = 1 SV = 1
MELSEPIVENGETEMSPEESWEHKEEISEAEPGGGSLGDGRPPEESAHEM
MEEEEEIPKPKSVVAPPGAPKKEHVNVVFIGHVDAGKSTIGGQIMYLTGM
VDKRTLEKYEREAKEKNRETWYLSWALDTNQEERDKGKTVEVGRAYFETE
KKHFTILDAPGHKSFVPNMIGGASQADLAVLVISARKGEFETGFEKGGQT
REHAMLAKTAGVKHLIVLINKMDDPTVNWSNERYEECKEKLVPFLKKVGE
NPKKDIHFMPCSGLTGANLKEQSDFCPWYIGLPFIPYLDNLPNENRSVDG
PIRLPIVDKYKDMGTVVLGKLESGSICKGQQLVMMPNKHNVEVLGILSDD
VETDTVAPGENLKIRLKGIEEEEILPGFILCDPNNLCHSGRTEDAQIVII
EHKSIICPGYNAVLHIHTCIEEVEITALICLVDKKSGEKSKTRPRFVKQD
QVCIARLRTAGTICLETFKDFPQMGRETLRDEGKTIAIGKVLKLVPEKD

In some cases, the reference protein is CiSPII and the reference amino acid motif begins at position 433 of SEQ ID NO: 12 and comprises 6, 7, 8, 9, 10, 11, 12, 13, 14, or 15 consecutive amino acids of SEQ ID NO: 12 (oriented N- to C- terminally from the beginning position). The first six amino acids, beginning at position 433 are underlined and bolded in SEQ ID NO: 12, above.

In some cases, the methods described herein include providing a three-dimensional structure of a reference protein. In some cases, the reference protein is GSPT1 and the three-dimensional structure is PDB entry GSPT1 PDB 5HXB or GSPT1 PDB 6XK6. In some cases, the reference protein is GSPT1 and the three-dimensional structure is “Eukaryotic peptide chain release factor GTP-binding subunit ERF3A, P15170, ERF3A_HUMAN”.

The coordinates of the Cα atoms of the reference amino acid motif beginning at position 433 of SEQ ID NO: 12, in PDB entry 5HXB chain A, amino acids 571-585 are as in Table E.

TABLE E
Coordinates for Motif in GSPT1

| Motif Position | Amino Acid | X | Y | X |
|---|---|---|---|---|
| X1 | D | −8.136 | 17.871 | 146.374 |
| X2 | K | −5.675 | 15.848 | 148.311 |
| X3 | K | −4.621 | 13.349 | 145.679 |
| X4 | S | −3.311 | 16.152 | 143.275 |
| X5 | G | −2.754 | 19.704 | 144.516 |
| X6 | E | −5.635 | 20.698 | 142.385 |
| X7 | K | −6.951 | 23.97 | 143.721 |
| X8 | S | −10.615 | 24.253 | 142.982 |
| X9 | K | −12.607 | 25.661 | 140.195 |
| X10 | T | −15.414 | 27.209 | 142.105 |
| X11 | R | −15.413 | 28.757 | 145.631 |
| X12 | P | −16.855 | 26.31 | 147.978 |
| X13 | R | −19.971 | 27.115 | 150.09 |
| X14 | F | −18.278 | 25.133 | 152.997 |
| X15 | V | −15.815 | 22.41 | 153.866 |

### Three-dimensional Characterization

In some cases, the methods described herein comprise three-dimensional characterization, e.g., of a test protein.

In some cases, the methods described herein comprise comparing three-dimensional structure(s), e.g., of a test protein and a reference protein. In some cases, the test protein comprises an amino acid motif described herein and the reference protein is a known substrate protein for cereblon.

In some cases, comparing the three-dimensional structure of a test protein and a reference protein comprises comparing the three-dimensional structure of the test protein's amino acid motif and the reference protein's corresponding amino acid motif, e.g., for structural similarity.

In some cases, the methods described herein comprise comparing the structural similarity of a test protein amino acid motif, e.g., as described herein, and a corresponding reference protein amino acid motif, e.g., as described herein for initially characterizing test protein(s) as a candidate cereblon substrate or not, and then optionally performing additional filtering to re-classify test protein(s) are candidate cereblon substrates or not. In some cases, comparing the structural similarity of the test protein amino acid motif and corresponding reference protein amino acid motif comprises calculating a BC score and/or an RMSD score, e.g., as described herein, while additional filtering is based on other characteristics, such as additional three-dimensional characterization, e.g., as described herein.

In some embodiments, the initial characterization includes both a structural similarity assessment and a structural context assessment, e.g., as described herein, while additional filtering is based on other characteristics, such as additional three-dimensional characterization, e.g., as described herein. In some cases, the structural context assessment comprises identifying whether the motif is present in a helix or not and, if not classifying the test protein as a candidate cereblon substrate, but if so, classifying the test protein as not a candidate cereblon substrate.

Thus, for example, in some cases, if the test protein has a BC score and/or RMSD score above a certain threshold (e.g., as defined herein), and is not found in a helix, then the test protein is classified as a candidate cereblon substrate and, optionally, additional filtering is applied, e.g., as described herein. If, on the other hand, for example, the test protein has a BC score and/or RMSD score above a certain threshold (e.g., as defined herein), and is found in a helix, then the test protein is classified as not a candidate cereblon substrate.

Three-dimensional structures can be obtained as described herein. In some instances, PDB entries are processed chain-wise and relevant information such as, but not limited to, amino acid boundaries, an amino acid sequence, and a secondary structure assignment by the PDB structure are extracted from the database.

In some embodiments, the method comprises assessing the similarity of the three-dimensional structure of the test protein, e.g., of a motif in a test protein as described herein, for structural similarity with a known degron structure, e.g., a G-loop of a known substrate protein for cereblon. In some embodiments, the known degron structure is selected from a database, such as a PDB database or AlphaFold2 database.

In some embodiments, assessing the similarity comprises modelling the structure of a modified protein, e.g., the amino acid sequence of a known substrate protein for cereblon that has been modified, e.g., computationally, to replace the known G-loop degron of the known substrate protein with a predicted degron amino acid sequence. For example, the three dimensional structure may be assessed by annotated PDB and/or recalculated from PDB parameters using a secondary structure assessment. In some instances, the secondary structure assessment comprises molecular visualization, e.g., using PyMOL (Schrödinger, L. & DeLano, W., 2020. PyMOL, Available at: pymol.org/pymol.).

In some embodiments, searching on the PDB database and manipulating of protein 3D models is performed with PyMOL. PyMOL is a user-sponsored molecular visualization system on an open-source foundation, maintained and distributed by Schrödinger. Other PDB database search engines and molecular visualization system are contemplated.

In some cases, three-dimensional characterization comprises determining atomic distances, structural context, structural similarity, cereblon binding compatibility, surface accessibility, and/or geometry, e.g., as described herein.

In some embodiments, the characterization comprises comparing a PyMOL secondary structure assignment, Binet-Cauchy kernel score (BC score), Clash Score, and/or surface accessibility calculation. In some embodiments, assessing comprises comparing a PyMOL secondary structure assignment and one or more of Binet-Cauchy kernel-based score (BC score), Clash Score, or surface accessibility score.

In some embodiments, the characterization is carried out using a scoring method selected from Binet-Cauchy kernel (BC score), SSAP (Orengo and Taylor, Methods Enzymol. 1996, 266, 617-635), DALI (Holm and Sander, Trends Biochem. Sci. 1995, 20, 478-480), CE (Shindyalov and Bourne, Protein Eng. 1998, 11, 739-747), MAMMOTH (Ortiz et al., Protein Sci. 2002, 11, 2606-2621), TM-align (Zhang and Skolnick, Nucleic Acids Res. 2005, 33, 2302-2309), root mean square deviation (RMSD) (Coutsias et al., J. Comput. Chem. 2004, 25, 1849-1857; Kabsch, Acta Cystallogr. 1976, 34, 827-828, Kabsch, Proteins, 1978, 37, 554-564), the unit-vector RMS distance (URMS) (Chew et al., J. Comput. Biol. 1999, 6, 313-325; Kedem et al., Proteins, 1999, 37, 554-564), the TM-score (Zhang and Skolnick, Proteins, 2004, 57, 702-710), Clash Score, and surface accessibility calculation, and any combination thereof

In some embodiments, characterization comprises a computer modeling and at least one similarity scoring method. In some embodiments, one or more scoring methods is used. In some embodiments, a combination of two, three, or more scoring methods is used.

In some embodiments, the computer modeling comprises comparing, e.g., using PyMOL, a secondary structure assignment with a known degron. In an example, the PyMOL assessment comprises (i) calculating the 3D structure coordinates of the amino acid positions of the predicted degron; (ii) comparing the coordinates to the 3D structure coordinates of a known or reference degron; and (iii) calculating a similarity score.

In some embodiments, the scoring method comprises a Binet-Cauchy kernel score (BC score). In some embodiments, the scoring method comprises a root mean square deviation score (RMSD). In some embodiments, the scoring method comprises a sequential structure alignment program score (SSAP). In some embodiments, the scoring method comprises protein structure comparison by distance alignment matrix method score (DALI). In some embodiments, the scoring method comprises protein structure alignment by incremental combinatorial extension score (CE). In some embodiments, the scoring method comprises MAtching Molecular Models Obtained from THeory score (MAMMOTH). In some embodiments, the scoring method comprises a template modeling alignment score (TM-align). In some embodiments, the scoring method comprises a template modeling score (TM-score). In some embodiments, the scoring method comprises the unit-vector root mean square (URMS) distance score. In some embodiments, the scoring method comprises a Clash Score. In some embodiments, the scoring method comprises a surface accessibility calculation.

### Structural Similarity Scoring Methods

In some cases, three-dimensional characterization comprises assessing structural similarity, e.g., between a test protein, e.g., a test protein motif described herein, and a reference protein, e.g., a reference protein described herein, e.g., a reference protein motif described herein.

Protein similarity searches can be performed at a global and a local level. Whole structure comparisons provide general information about protein classification and protein functions. At a more local level, fragment comparison and identification has become a key step for protein structure analysis, annotation and modeling. Fragment similarities reveal functionally important residues (Tendulkar et al., PLoS One, 2010, 5, e9678), similar structural motifs may indicate function preservation in remote homologs (Manikandan et al., Genome Biol. 2008, 9, R52), and more generally, recurring fragments may be used as building blocks to the construction of de novo models of protein structures (Bystroff et al., Curr. Opin. Biotechnol. 1996, 7, 417-421; Friedberg and Godzik, Structure, 2005, 12, 1213-1224; Samson and Levitt, Nucleic Acid Res. 2009, 37, D224; Unger et al., Proteins, 1989, 5, 355-373). Meaningful scores to assess protein structure similarity are essential to decipher protein structure and sequence evolution. The mining of the increasing number of protein structures requires fast and accurate similarity measures with statistical significance.

In some cases, the structural similarity is assessed using a BC score and/or a RMSD score.

### BC Score

In the context of protein structure comparison, the BC score (see, e.g., Guyon et al., “Fast Protein Fragment Similarity Scoring Using a Binet-Cauchy Kernel,” Bioinformatics 30(6):784-91 (2014)) is a shape similarity score corresponding to a correlation score between fragment shapes. Hence, it is normalized and its values range from −1 measuring perfect shape anti-similarity (one fragment is the minor image of the second one) to 1 indicating perfect similarity (up to a linear deformation). The BC score is independent of any rotation to the structures and consequently its computation does not involve a prior superimposition of the structures. The score is relatively fast to compute requiring the computation of 3×3 matrix determinants. Therefore, it is well adapted to perform large-scale protein mining and is designed to compare short protein fragments.

In one example, the scoring method comprises a BC score. The BC score is a cosine normalized similarity score: 1 is a perfect match, 0 for a completely dissimilar match, and -1 for a minor image. Matched amino acids segments are scored with a Binet-Cauchy kemel-based score (BC score) on the Cα positions of the protein segment (Guyon and Tuffery, Bioinformatics, 30(6), 784-791) using the formula (1):

$$BC ( X , Y ) = \frac{det ( {X}^{τ} Y )}{\sqrt{det ( {X}^{τ} X ) det ( {Y}^{τ} Y )}} . \qquad ( 1 )$$

The BC score can be further normalized and tuned to account for distance constraints.

In some cases, the BC score is calculated by comparing the three-dimensional coordinates of the Cα atoms for each amino acid in a test protein amino acid motif, e.g., as described herein, (X in the formula above) and for each amino acid in a reference amino acid motif, e.g., as described herein (Y in the formula above).

In some embodiments, the BC score is a first scoring method. In some embodiments, the BC score is at least about 0.50. In some embodiments, the BC score is at least about 0.60.

In some embodiments, the BC score is from about 0.50 to about 1. In some embodiments, the BC score is about 0.50. about 0.55, about 0,60, about 0,65, about 0,70, about 0.75, about 0.80, about 0,85, about 0,90, about 0,95, or about 1. In some embodiments, the BC score is about 0.80, about 0.81, about 0.82, about 0.83, about 0.84, about 0.85, about 0.86, about 0.87, about 0.88, about 0.89, about 0.90, about 0.91, about 0.92, about 0.93, about 0.94, about 0.95, about 0.96, about 0.97, about 0.98, or about 0.99. In some embodiments, the BC score is about 0.85. In some embodiments, the BC score is about 0.86. In some embodiments, the BC score is about 0.87. In some embodiments, the BC score is about 0.88. In some embodiments, the BC score is about 0,89. In some embodiments, the BC score is about 0.90. In some embodiments, the BC score is about 0.91. In some embodiments, the BC score is about 0.92. In some embodiments, the BC score is about 0.93. In some embodiments, the BC score is about 0.94. In some embodiments, the BC score is about 0,95. In some embodiments, the BC score is about 096. In some embodiments, the BC score is about 0.97. In some embodiments, the BC score is about 0.98. In some embodiments, the BC score is about 0.99.

In some embodiments, the BC-score is compared with a RMSD score. In some embodiments, the RMSD score is substantially similar to the BC-score.

In some embodiments, the RMSD score is further used to calculate a p-value and a clash RMSD score.

In some embodiments, one or more scoring methods are used after an initial BC-score is assessed.

### RMSD Score

In some cases, the structural similarity is assessed using a root mean square deviation (RMSD) score, e.g., as described in Coutsias et al., J. Comput. Chem. 2004, 25, 1849-1857; Kabsch, Acta Cystallogr. 1976, 34, 827-828, Kabsch, Proteins, 1978, 37, 554-564), and/or a unit-vector RMS distance (URMS), e.g., as described in Chew et al., J. Comput. Biol. 1999, 6, 313-325; Kedem et al., Proteins, 1999, 37, 554-564.

### Structural Context

In some cases, three-dimensional characterization comprises determining the structural context, e.g., the secondary structural context of the amino acid motif in the candidate substrate protein for cereblon. In some cases, determining the structural context comprises determining whether the motif is or is not in a loop, helix, and/or strand, e.g., based on a three-dimensional structure, e.g., a classification from a PDB and/or AlphaFold2 database entry.

In some embodiments, the predicted degron is not in a helix of the identified protein. In some embodiments, the predicted degron is not in an α-turn of the identified protein. In other embodiments, the predicted degron in not in a β-hairpin of the identified protein.

### Atomic Distances

In certain embodiments, three-dimensional characterization comprises assessing differences in atomic distances, e.g., within a motif of a test protein as described herein.

In some embodiments, a distance from amino acid position X¹ to X⁴ is from about 1 to about 10 angstroms. In some embodiments, a distance from amino acid position X¹ to X⁴ is from about 5 to about 10 angstroms. In some embodiments, a distance from amino acid position X¹ to X⁴ is less than about 5, about 6, about 7, about 8, about 9, or about 10 angstroms. In some embodiments, a distance from amino acid position X¹ to X⁴ is less than about 5 angstroms. In some embodiments, a distance from amino acid position X¹ to X⁴ is less than about 6 angstroms. In some embodiments, a distance from amino acid position X¹ to X⁴ is less than about 7 angstroms. In some embodiments, a distance from amino acid position X¹ to X⁴ is less than about 8 angstroms. In some embodiments, a distance from amino acid position X¹ to X⁴ is less than about 9 angstroms. In some embodiments, a distance from amino acid position X¹ to X⁴ is less than about 10 angstroms.

### Cereblon Binding Compatibility

### Clash Score

In an example, the scoring assessment comprises a Clash Score. The clash score is a numerical indication of how many pairs of atoms are unusually close together and depends on the protein domain structure, flexibility, and globularity. The clash score is a measure of protein binding compatibility to cereblon (Matsumoto, S. et al. Nature 2019, 571(7763), 79-84 and Michael, A. et al. Science, 2020, 368(6498), 1460-1465.

In some cases, the clash score is calculating using the entire three-dimensional structure of the test protein as input. In some cases, the clash score is calculated using a portion of the three-dimensional structure of the test protein as input, e.g., the three-dimensional structure of one or more domains of the test protein.

In some embodiments, the clash score comprises a clash_atm_count or class_aa_count. The clash_atm_count is a measure of atom overlapping of the entire parent chain on superposition of a candidate G-loop to cereblon based on superposition with a G-loop degron complex with cereblon. A clash_aa_count is similar to a clash_atm_count; it however relies on the number of amino acids instead of the number of atoms. Candidate substrate atoms that are within in 1 Å (angstrom) of an atom in cereblon are scored.

In some embodiments, the clash_atm_count is correlated with the clash_aa_count. In some instances, the clash_aa_count is lower than the clash_atm_count.

In some embodiments, the clash_atm_count is less than about 10. In some embodiments, the clash_aa_count is less than about 8.

### Glycine Superposition

In some embodiments, the clash score comprises glycine_super_dist. The glycine distance is the interatomic distance between Cα atoms defined by a key position index which defaults to the 5 position in the G-loop sequence. In some embodiments, the glycine_super_dist is calculated if the BC-score is >0.6. In some embodiments, the glycine_super_dist is <1 Å.

In some embodiments, the clash score comprises a clash_rmsd (root mean square derivation). The root-mean-square deviation of backbone atoms of the candidate G-loop and reference degron is correlated to be correlated with Cα atom structural comparison scores BC-score. Lower clash_rmsd scores are favored. In some embodiments the clash_rsmd is less than about 1.5 Å².

### Surface Accessibility

In some embodiments, the scoring comprises a surface accessibility calculation. The accessible surface area or solvent-accessible surface area is the surface area of a biomolecule that is accessible to a solvent. Both methods assess interactions between protein surfaces.

In some embodiments, the surface accessibility calculation comprises surface_exposure which is a measure of surface accessibly. In some instances, the threshold is 2.50 (score for a match in a buried α-helix). Scores for known G-loop degrons range from about 2.8 to about 3.5. In some embodiments, the score is a sum of the surface exposure of each amino acid in the 6 amino acid candidate G-loop wherein, integer 1 is exposed and 0 is buried. In some embodiments, the surface_exposure is great than about 2.5

In some embodiments, the surface accessibility is normalized. In some embodiments the threshold is >0.35 (score for a match in a buried α-helix). Typical scores for known G-loop degrons range from about 0.40 to about 0.55. In some embodiments, surface_exposure_normalized is calculated only if the BC score is >0.6. In some embodiments, the surface_exposure_normalized is great than about 0.35.

In some embodiments, the surface accessibility calculation comprises calculating neighbouring_atm_count_chain This is a measure of the crowing and/or isolation of a candidate G-loop in its parent chain. In some embodiments, neighboring atoms within 4 A are counted. In some embodiments, neighbouring_atm_count_chain is assessed if a BC-score>0.6.

In some embodiments, the surface accessibility calculation comprises calculating neighbouring_atm_countbiomt. This is a measure of crowding and/or isolation in the biological assembly in the parent complex if defined. In some embodiments, neighbouring_atm_count_biomt is assessed if a BC-score>0.6.

### Geometry

In some embodiments, scoring comprises assessing a loop_restrictive_distance. This is defined as the interatomic distance between Cα atoms start and end amino acids (X¹ and X⁵) of a candidate G-loop. The loop_restrictive_distance threshold is <7 Å as formally defined for protein α-turns (5 amino acids), which includes G-loop degrons. In some embodiments, loop_restrictive_distance is assessed if a BC-score>0.6. In some embodiments, loop_restrictive_distance is less than about 7.

### Classification

In some cases, the methods described herein comprise classifying and/or re-classifying a test protein(s) as a candidate substrate protein for cereblon, e.g., based on one or more three-dimensional characterization, as described herein. In some cases, after an initial classification, further optional filtering criteria, e.g., based on one or more three-dimensional characterization(s), e.g., as described herein, are used to re-classify a test protein(s) as a candidate substrate for cereblon.

In some cases, a scoring assessment is carried out based on three-dimensional characterization, e.g., as described herein. In some cases, the characterization is based on one or more scores, e.g., as described herein. In some cases, a test protein is characterized and/or re-characterized as a candidate cereblon substrate if a key condition is met for one or more of the assessed scores.

In some embodiments, the scoring assessment comprises performing the assessments described in Table 1 and/or Table 2. In some cases, the test protein(s) are characterized as candidate cereblon substrate(s) if one or more of the conditions shown in Table 1 and/or Table 2 is met. In some cases, initial characterization is based on similarity score, e.g., a bc_score, e.g., as described herein.

In some cases, the test protein(s) are characterized and/or re-characterized as candidate cereblon substrates if the bc_score is 0.5 or more, 0.55 or more, 0.6 or more, 0.65 or more, 0.7 or more, 0.75 or more, 0.8 or more, 0.85 or more, 0.9 or more, or 0.95 or more.

In some cases, the test protein(s) are characterized and/or re-characterized as candidate cereblon substrates if the secondary structure (e.g., as calculated by secondary_structure_pdb and/or secondary_structure_pymol), is not a helix.

In some cases, the test protein(s) are characterized and/or re-characterized as candidate cereblon substrates based on a cereblon binding compatibility score. In some cases, the cereblon binding capacity score is a clash score. In some cases, the clash score is clash_rmsd. In some cases, the clash score is clash_atm_count. In some cases, the clash score is clash_aa_count. In some cases, the clash score is glycine_super_dist. In some cases, the clash score is glycine_super_dist_ok.

In some cases, the test protein(s) are characterized and/or re-characterized as candidate cereblon substrates based on a local accessibility/isolation score. In some cases, the local accessibility/isolation score is surface_exposure. In some cases, the local accessibility/isolation score is surface_exposure_normalized. In some cases, the local accessibility/isolation score is neighbouring_atm_count_chain. In some cases, the local accessibility/isolation score is neighbouring_atm_count_biomt.

In some cases, the test protein(s) are characterized and/or re-characterized as candidate cereblon substrates based on a geometry score. In some cases, the geometry score is loop_restrictive_distance.

In some cases, the test protein(s) are characterized and/or re-characterized as candidate cereblon substrate if the loop_restrictive_distance score is

In some embodiments, scoring is performed by performing the following representative assessment(s) described in Table 1.

TABLE 1
Scoring Assessment Version 1

| Assessment | Score | Condition | Description |
|---|---|---|---|
| Initial | bc_score | >0.5 | Initial structural similarity score to a known |
| structural | | | G-loop degron reference (Cα atoms only). A |
| comparison | | | Binet-Cauchy kernel-based cosine |
| | | | normalized similarity score (valid range −1 to |
| | | | 1). Threshold > 0.5. Range for good scores |
| | | | 0.85-1.00. A primary score |
| | bc_score_rmsd | | Root-mean-square deviation lower the better |
| | | | (Å²). “bc_score” preferred. Calculated at the |
| | | | same time as the “bc_score”. Useful to |
| | | | compare to “clash_rmsd” in some cases - |
| | | | similar, but non-identical values are expected. |
| | | | A secondary score |
| | bc_score_pval | | P-value derived from “bc_score” calculation. |
| | | | A secondary score |
| Structural | secondary_structure_pdb | != H | Secondary structure annotations for a |
| context | | | candidate G-loop (from PDB: ‘L’ = loop, ‘’ = |
| | | | loop, ‘H’ = helix, ‘S’ = strand). Always |
| | | | extracted. A primary score |
| | secondary_structure_pymol | != H | Secondary structure annotations for the |
| | | | candidate G-loop (reassigned in PyMOL |
| | | | using “DSS”: L = loop, empty string = loop, |
| | | | H = helix, S = strand). Always extracted. A |
| | | | primary score |
| Cereblon | clash_rmsd | | Root-mean-square deviation of backbone |
| binding | | | atoms of candidate G-loop and reference |
| compatibility | | | model (Å²). Expected to be correlated with |
| | | | Cα atom structural comparison scores: |
| | | | “bc_score” and “bc_score_rms”. Lower is |
| | | | better. Range ill-defined and calculation can |
| | | | be unstable. <1.5 Å² is reasonable |
| | | | conditional. Only calculated if “bc_score” > |
| | | | 0.5. A secondary score |
| | clash_atm_count | <10 | Atom count. A measure of atom overlapping |
| | | | of entire parent chain on superposition of a |
| | | | candidate G-loop to cereblon based on |
| | | | superposition with a G-loop degron complex |
| | | | with cereblon (from PDB) - number of |
| | | | candidate neosubstrate atoms that are within |
| | | | in 1 Å of an atom in cereblon. Rank very low |
| | | | values highly, but do not rule out candidates |
| | | | with slightly elevated |
| | | | ‘clash_atm_count scores' if other key scores |
| | | | fall within the acceptable range as defined |
| | | | here. Expected to depend on protein domain |
| | | | structure, flexibility, and globularity. Only |
| | | | calculated if “bc_score” > 0.5. A primary |
| | | | score |
| | clash_aa_count | <8 | Correlated with “clash_atm_count”, but with |
| | | | a presumably lower count. Count of parent |
| | | | amino acid residues rather than individual |
| | | | atoms. Only calculated if “bc_score” > 0.5. A |
| | | | secondary score |
| Local | surface_exposure | >2.5 | Measure of surface accessibly. Threshold |
| accessibility/ | | | 2.50 (score for a match in a buried α-helix). |
| isolation | | | Only calculated if “bc_score” > 0.5. Scores |
| | | | for known G-loop degrons range from 2.8 to |
| | | | 3.5. Score is a sum of the surface exposure of |
| | | | each amino acid in the 6 aa candidate G-loop |
| | | | (1 being exposed/0 being buried). A |
| | | | primary score |

In another non-limiting embodiment, scoring is performed by performing the following representative assessment(s) described in Table 2.

TABLE 2
Scoring Assessment Version 2

| | | Key | |
|---|---|---|---|
| Assessment | Score | condition | Description |
| Initial | bc_score | >0.6 | Initial structural similarity score to a known |
| structural | | | G-loop degron reference (Cα atoms only). A |
| comparison | | | Binet-Cauchy kernel-based cosine |
| | | | normalized similarity score (valid range −1 |
| | | | to 1). Threshold > 0.6. Range for good |
| | | | scores 0.85-1.00. A primary score |
| | bc_score_rmsd | | Root-mean-square deviation lower the |
| | | | better (Å²). “bc_score” preferred. |
| | | | Calculated at the same time as the |
| | | | “bc_score”. Useful to compare to |
| | | | “clash rmsd” in some cases - similar, but |
| | | | non-identical values are expected. A |
| | | | secondary score |
| Structural | secondary_structure_pdb | != H | Secondary structure annotations for a |
| context | | | candidate G-loop (from PDB: ‘L’ = loop, |
| | | | ‘’ = loop, ‘H’ = helix, ‘S’ = strand). Always |
| | | | extracted. A primary score |
| | secondary_structure_pymol | != H | Secondary structure annotations for the |
| | | | candidate G-loop (reassigned in PyMOL |
| | | | using “DSS”: L = loop, empty string = loop, |
| | | | H = helix, S = strand). Always extracted. A |
| | | | primary score |
| Cereblon | clash_rmsd | | Root-mean-square deviation of backbone |
| binding | | | atoms of candidate G-loop and reference |
| compatibility | | | model (Å²). Expected to be correlated with |
| | | | Cα atom structural comparison |
| | | | scores: “bc_score” and “bc_score_rms”. |
| | | | Lower is better. Range currently ill-defined |
| | | | and calculation can be unstable. <1.5 Å² is |
| | | | reasonable conditional |
| | | | on “glycine_super_dist_ok” being “True”. |
| | | | Only calculated if “bc_score” > 0.6. A |
| | | | secondary score |
| | clash_atm_count | <10 | Atom count. A measure of atom |
| | | | overlapping of entire parent chain on |
| | | | superposition of a candidate G-loop to |
| | | | cereblon based on superposition with a G- |
| | | | loop degron complex with cereblon (from |
| | | | PDB) - number of candidate neosubstrate |
| | | | atoms that are within in 1 Å of an atom in |
| | | | cereblon. Conditional |
| | | | on “glycine_super_dist_ok” being “True”. |
| | | | Rank very low values highly, but do not |
| | | | rule out candidates with slightly elevated |
| | | | ‘clash_atm_count scores' if other key scores |
| | | | fall within the acceptable range as defined |
| | | | here. Expected to depend on protein domain |
| | | | structure, flexibility, and globularity. Only |
| | | | calculated if “bc_score” > 0.6. A primary |
| | | | score |
| | clash_aa_count | <8 | Correlated with “clash_atm_count”, but |
| | | | with a presumably lower count. Count of |
| | | | parent amino acid residues rather than |
| | | | individual atoms. Only calculated if |
| | | | “bc_score” > 0.6. A secondary score |
| | glycine_super_dist | <1 | Interatomic distance between Cα atoms |
| | | | defined by a key position index (defaults to |
| | | | 5 for G-loops) in the superposition used for |
| | | | “clash_atm_count” and “clash_aa_count” |
| | | | calculation. Restrictive distance < 1 Å. Only |
| | | | calculated if “bc_score” > 0.6. A primary |
| | | | score |
| | glycine_super_dist_ok | True | True if “glycine_super_dist” is below the |
| | | | restrictive distance (<1 Å). Only calculated |
| | | | if “bc_score” > 0.6. A primary score |
| Geometry | loop_restrictive_distance | <7 | Interatomic distance between Cα atoms start |
| | | | and end amino acids (i and i + 4) of a |
| | | | candidate G-loop (Å). Threshold < 7 Å - |
| | | | restrictive distance as formally defined for |
| | | | protein α-turns (5 amino acids), which |
| | | | includes G-loop degrons. Only calculated if |
| | | | “bc_score” > 0.6. A secondary score |
| Local | surface_exposure_normalised | >0.35 | Measure of surface accessibly normalized. |
| accessibility/ | | | Threshold > 0.35 (score for a match in a |
| isolation | | | buried α-helix). Only calculated if |
| | | | “bc_score” > 0.6. Scores for known G-loop |
| | | | degrons range from 0.40 to 0.55. A primary |
| | | | score |
| | neighbouring_atm_count_chain | | Measure of crowding/isolation of |
| | | | candidate G-loop in its parent chain. Count |
| | | | of neighboring atoms within 4 Å. Lower is |
| | | | better. Only calculated if “bc_score” > 0.6. |
| | | | A secondary score |
| | neighbouring_atm_count_biomt | | Measure of crowding/isolation in the |
| | | | biological assembly (parent complex) if |
| | | | defined. See |
| | | | “neighbouring_atm_count_chain” |
| | | | definition. Lower is better. Only calculated |
| | | | if “bc score” > 0.6. A secondary score |

### Further Characterization

In some embodiments provided herein, the methods further comprise testing the identified candidate degron-containing substrate protein, e.g., in a substrate detection assay such as a cereblon-mediated degradation assay, ubiquitination assay, or proteomics experiment.

In some embodiments provided herein, the methods further comprise testing the identified candidate degron-containing substrate protein in a cereblon-mediated degradation assay. In some embodiments, the methods further comprise testing the identified candidate degron-containing substrate protein in a cereblon-mediated degradation assay with a small molecule compound that binds to cereblon (i.e. a degrader compound) and/or cereblon modifying agent.

In some embodiments, the method further comprises (i) testing the candidate protein in a cereblon-mediated assay with a degrader compound; and (ii) measuring the protein levels.

In some embodiments provided herein, the methods further comprise testing the identified candidate degron-containing substrate protein in a ubiquitination assay. In some embodiments, the methods further comprise testing the identified candidate degron-containing substrate protein in a ubiquitination assay in the presence of a degrader compound.

In some embodiments provided herein, the methods further comprise testing the identified candidate degron-containing substrate protein in a proteomics experiment. In some embodiments, the methods further comprise testing the identified candidate degron-containing substrate protein in a proteomics experiment in the presence of a degrader compound.

In some embodiments, the identified candidate degron-containing substrate protein for cereblon is further characterized by being bound to a cereblon modifying compound or agent that alters the 3-D structure of cereblon. In some embodiments, the modifying agent induces a cereblon conformational change (e.g., within the binding pocket of the cereblon) or otherwise alters the properties of a cereblon surface. In some embodiments, the candidate substrate protein induces a conformational change in the 3-D structure of cereblon.

### Substrate Detection Assays

In some cases, the methods described herein comprise testing or having tested candidate degron-containing substrate protein(s), in an E3 ligase substrate detection assay. In some cases, the assay is carried out in the absence of a binding modulator of the E3 ligase. In some cases, the assay is carried out in the presence of a binding modulator of the E3 ligase.

E3 ligase substrate detection assays are described, for example, in Liu et al., “Assays and Technologies for Developing Proteolysis Targeting Chimera Degraders,” Future Medicinal Chemistry 12(12):1155-79 (2020).

E3 ligase substrate detection assays include, for example, binding/ternary binding affinities and ternary complex formation assays used to profile, for example, ternary complex formation, population, stability, binding affinities, cooperative or kinetics such as fluorescence polarization (FP) assay, an amplified luminescent proximity homogenous assay (ALPHA), time-resolved fluorescence energy transfer assay (TR-FRET), isothermal titration calorimetry (ITC), surface plasma resonance (SPR), bio-layer interferometry (BLI), nano-bioluminescence resonance energy transfer (nano-BRET), size exclusive chromatography (SEC), crystallography, co-immunoprecipitation (Co-IP), mass spectrometry (MS), and protein-fragment complementation (e.g., NanoBiT®). See, e.g., Liu et al., 2020.

E3 ligase substrate detection assays include, for example, protein ubiquitination assays. See, e.g., Liu et al., 2020.

E3 ligase substrate detection assays include, for example, target degradation assays such as immunoassays, reporter assays, mass spectrometry (MS), protein degradation-based phenotypic screening such as amplified luminescent proximity homogenous assay (ALPHA), bio-layer interferometry (BLI), cellular thermal shift assay (CETSA), co-immunoprecipitation (Co-IP), cryogenic electron microscopy (Cryo-EM), differential scanning fluorimetry (DSF), fluorescence polarization (FP), isothermal titration calorimetry (ITC), microscale thermophoresis (MST), NanoLuc binary technology (Nano-BiT), nano-bioluminescence resonance engery transfer (BRET), surface plasma resonance (SPR), time-resolved fluorescence energy transfer (TR-FRET), tandem ubiquitin-binding entities-amplified luminescent proximity homogenous and enzyme-linked immunosorbent assay (TUBE-ALPHALISA), and tandem ubiquitin-binding entities-dissociation-enhanced lanthanide fluorescent immunoassay (TUBE-DELFIA). See, e.g., Liu et al., 2020.

In some cases, the E3 ligase substrate detection assay is a proximity assay. In some cases, the E3 ligase substrate detection assay is a binding assay. In some cases, the E3 ligase substrate detection assay is a degradation assay.

In some cases, the proximity assay is a homogeneous time resolved fluorescence (HTRF) assay. In some cases, the proximity assay is a quantitative proteomics assay. In some cases, the proximity assay is a biotinylation assay, e.g., a promiscuous biotinylation assay.

In some cases, the degradation assay is a High efficiency Binary Technology (HiBiT) assay.

In some cases, the degradation assay is a quantitative proteomics assay.

In some cases, the E3 ligase substrate detection assay is a yeast-2-hybrid system. See, e.g., Kohalmi et al., “Identification and Characterization of Protein Interactions Using the Yeast-2-Hybrid System,” In: Gelvin S. B., Schilperoort R. A. (eds) Plant Molecular Biology Manual. Springer, Dordrecht (1998).

In some cases, the E3 ligase substrate detection assay is a genomic construct based method, e.g., as described in Sievers et al., “Defining the Human C2H2 Zinc Finger Degrome Targeted by Thalidomide Analogs through CRBN,” Science 362(6414):eaat0572 (2018).

In some cases, the E3 ligase substrate detection assay is an indirect screen, e.g., to detect changes in gene and/or protein expression.

### Candidate Degron Binding

In some embodiments, the binding of the candidate substrate protein and cereblon is characterized, either in the presence of an E3 ligase binding modulator or in the absence of an E3 ligase binding modulator.

In some embodiments, one or more additional residues in cereblon forms a non-covalent interaction with the degron. In some instances, the non-covalent interaction is a hydrophobic interaction, charged interaction (e.g., either positively charged or negatively charged interaction), polar interaction, H-bonding, salt bridge, pi-pi stacking, or cation-pi interaction. In some embodiments, one or more amino acids of the degron form interactions with one or more amino acids selected from a group consisting of the amino acid residues 150, 352, 353, 355, 357, 377, 380, 386, 388, 397, and 400 of isoform 1 of human cereblon. In some embodiments, the interaction is a hydrogen bond. In other embodiments, the interaction is a Van der Waals interaction.

In some embodiments, one or more amino acids of the degron form hydrogen bonds with one or more amino acids within amino acid residues 300-450 of cereblon. In some embodiments, one or more amino acids of the degron form hydrogen bonds with one or more amino acids within amino acid residues 350-430 of cereblon. In some embodiments, one or more amino acids of the degron form hydrogen bonds with one or more amino acids within amino acid residues 351-422 of cereblon. In some embodiments, one or more amino acids of the degron form hydrogen bonds with one or more amino acids within amino acid residues 351-357 of cereblon. In some embodiments, one or more amino acids of the degron form hydrogen bonds with one or more amino acids within amino acid residues 377-400 of cereblon. In some embodiments, the cereblon is the isoform 1 of cereblon. In other embodiments, the cereblon is the isoform 2 of the cereblon. In some embodiments, the cereblon is the human cereblon.

In some embodiments, the amino acid residues at any of position of the degron form hydrogen bonds with amino acid residues on cereblon. In some embodiments, the amino acid residues at position X¹, X², X³, . . . , or X⁶ form hydrogen bonds with amino acid residues on cereblon. In some embodiments, the amino acid residues at position X¹, X², or X³ form hydrogen bonds with amino acid residues on cereblon. In some embodiments, the amino acid residues at positions X¹ and X² form hydrogen bonds with amino acid residues on cereblon. In some embodiments, the amino acid residue at position X¹ form hydrogen bonds with amino acid residues on cereblon. In another embodiment, the amino acid residue at position X² form hydrogen bonds with amino acid residues on cereblon. In some embodiments, the amino acid residue at position X³ form hydrogen bonds with amino acid residues on cereblon.

### Cereblon Binding Modulators

The methods described herein are useful, for example, for identifying target substrates that interact with cereblon, e.g., selectively, e.g., in the presence of a compound, e.g., an E3 ligase binding modulator, e.g., a cereblon binding modulator. In some cases, the E3 ligase binding modulator is a targeted protein degrader.

E3 ligase binding modulators, e.g., cereblon binding modulators, including targeted protein degraders, are described, for example, in WO2021/069705 and WO2021/053555, which are hereby incorporated by reference in their entirety.

### Predicted Degrons

In another aspect, provide herein is a predicted degron identified by any of the methods described herein, e.g., for use in identifying a candidate substrate of cereblon. In some cases, use in identifying a candidate substrate of cereblon is carried out according to any of the methods described herein.

In some embodiments, the predicted degron is identified by computational methods. In some embodiments, the predicted degron is further characterized and/or confirmed by protein degradation or binding assays.

In some embodiments, the predicted degron comprises an amino acid sequence of about 5 to about 15 amino acids in length, 6 to about 12 amino acids in length, at least about 6 amino acids, at least about 7 amino acids, at least about 8 amino acids, at least about 9 amino acids, or at least about 10 amino acids.

In some embodiments, the predicted degron comprise a glycine (G) in the 5 amino acid position.

In some embodiments, the predicted degron is in a G-loop of a candidate substrate protein.

In some embodiments, the candidate substrate protein(s) for cereblon comprising the predicted degron(s) described herein are substrate proteins targeted for degradation by the E3 ligase machinery. In some embodiments, the candidate substrate protein(s) for cereblon comprising the predicted degron(s) described herein are substrate proteins targeted for selective degradation by the E3 ligase machinery, e.g., in the presence of an E3 ligase binding modulator, e.g., as described herein.

In some embodiments, the candidate substrate protein(s) for cereblon comprising the predicted degron(s) described herein are protein substrate(s) of the E3 ubiquitin ligase complex comprising cereblon bound to a small molecule compound described herein.

### Definitions

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The terms “cereblon” or “CRBN” and similar terms refers to the polypeptides (“polypeptides,” “proteins” are used interchangeably herein) comprising the amino acid sequence of any cereblon, such as a human cereblon protein (e.g., human CRBN isoform 1, GenBank Accession No. NP_057386 (SEQ ID NO: 1); or human cereblon isoform 2, GenBank Accession No. NP 001166953 (SEQ ID NO: 2), or SEQ ID NO: 3, SEQ ID NO: 4, SEQ ID NO: 5, SEQ ID NO: 6, or SEQ ID NO: 7, each of which is herein incorporated by reference in its entirety), and related polypeptides, including SNP variants thereof Related cereblon polypeptides include allelic variants (e.g., SNP variants); splice variants; fragments; derivatives; substitution, deletion, and insertion variants; fusion polypeptides; and interspecies homologs, which, in certain embodiments, retain cereblon activity, e.g., ability to ubiquinate substrate protein(s), whether in the presence or absence of an E3 ligase binding modulator.

The term “cereblon modifying agent” refers to a molecule that directly or indirectly modulates the cereblon E3 ubiquitin-ligase complex. In some embodiments, the modifying agent can bind directly to cereblon and induce conformational change in the cereblon protein. In other embodiments, the modifying agent can bind directly to other subunits in the cereblon E3 ubiquitin-ligase complex.

As used herein the terms “polypeptide” and “protein” are interchangeable and as used herein, refer to a polymer of amino acids of three or more amino acids in a serial array, linked through peptide bonds. The term “polypeptide” includes proteins, protein fragments, protein analogues, oligopeptides and the like. The term polypeptide as used herein can also refer to a peptide. The amino acids making up the polypeptide may be naturally derived, or may be synthetic. The polypeptide can be purified from a biological sample.

As used herein, the amino acids, which occur in the various amino acid sequences appearing herein, are denoted by their well-known, three-letter or one-letter abbreviations.

As used herein, amino acid residue refers to an amino acid formed upon chemical digestion (hydrolysis) of a polypeptide at its peptide linkages. The amino acid residues described herein are, in certain embodiments, in the “L” isomeric form. Residues in the “D” isomeric form can be substituted for any “L” amino acid residue, as long as the desired functional property is retained by the polypeptide. —NH₂ refers to the free amino group present at the amino terminus of a polypeptide. —CO₂H refers to the free carboxy group present at the carboxyl terminus of a polypeptide. In keeping with standard polypeptide nomenclature described in J. Biol. Chem., 243:3552 59 (1969) and adopted at 37 C.F.R. §§ 1.821-1.822, abbreviations for amino acid residues are shown in the following Table 3:

TABLE 3
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| Z | Glx | Glu and/or Gln |
| W | Trp | tryptophan |
| R | Arg | arginine |
| D | Asp | aspartic acid |
| N | Asn | asparagine |
| B | Asx | Asn and/or Asp |
| C | Cys | cysteine |
| X | Xaa | Unknown or other |

It should be noted that all amino acid residue sequences represented herein by formulae have a left to right orientation in the conventional direction of amino terminus to carboxyl terminus. In addition, the phrase “amino acid residue” is broadly defined to include the amino acids listed in the Table of Correspondence and modified and unusual amino acids, such as those referred to in 37 C.F.R. §§1.821-1.822, and incorporated herein by reference. Furthermore, it should be noted that a dash at the beginning or end of an amino acid residue sequence indicates a peptide bond to a further sequence of one or more amino acid residues or to an amino terminal group such as —NH₂ or to a carboxyl terminal group such as —CO₂H.

In a peptide or protein, suitable conservative substitutions of amino acids are known to those of skill in this art and can be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions can be made in accordance with those set forth in Table 4 as follows:

TABLE 4

| Original residue | Conservative substitution |
|---|---|
| Ala (A) | Gly; Ser |
| Arg (R) | Lys |
| Asn (N) | Gln; His |
| Asp (D) | Glu |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and can be determined empirically or in accord with known conservative substitutions. In some embodiments, although conservative amino acid substitutions may be possible in the degrons described herein, the glycine at position 5 (i.e X⁵) is critical and is not altered.

The terms below, as used herein, have the following meanings, unless indicated otherwise:

The section headings used herein are for organizational purposes only and are not to be construed as limiting the subject matter described.

### EXAMPLES

The invention is further described in the following examples, which do not limit the scope of the invention described in the claims.

### Example 1: In silico Analysis and Cataloguing of G-loop Containing Proteins

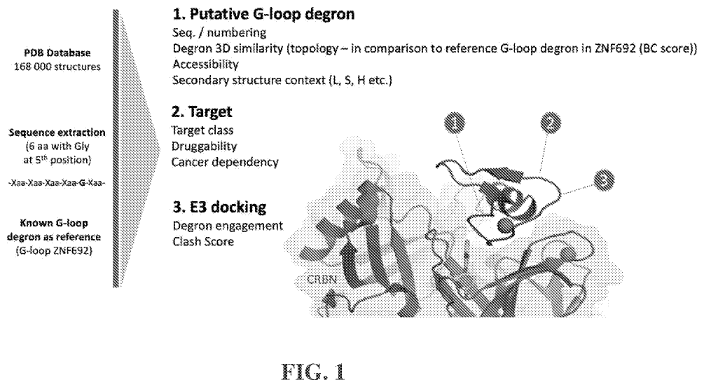

A non-limiting method of a computational discovery process for an in silico analysis searching of proteins containing G-loops is shown in FIG. 1. G-loop containing proteins are catalogued as potential protein substrates for cereblon.

### Validation of Discovery Process

A non-limiting method for computationally searching for a protein based on the structural similarity to a known degron is described.

Python using the PyMOL API. The algorithm of this computational method used the following 5 steps:

Step 1. Protein database PDB entries are processed chain-wise.

Step 2. Motif search of each chain's amino acid sequence (regular expression matching) -6 amino acid (aa) pattern with a Gly in the 5th position (‘X¹—X²—X³—X⁴-G-X⁶’). In some embodiments, 8 amino acid (aa) pattern with a Gly in the 5th position (‘X¹—X²—X³—X⁴-G—X⁶—X⁷—X⁸’) was used. In some other embodiments, 10 amino acid (aa) pattern with a Gly in the 5th position (‘X¹—X²—X³—X⁴—X⁵—X⁶—X⁷—X⁸—X⁹—X¹⁰’) was used. In some other embodiments, 12 amino acid (aa) pattern with a Gly in the 5th position (‘X¹—X²—X³—X⁴—X⁵—X⁶—X⁷—X⁸—X⁹—X¹⁰—X¹¹—X¹²’) was used.

Step 3. Extract relevant information for a match: amino acid sequence boundaries; amino acid sequence; secondary structure assignment as annotated by the PDB, and recalculated using a more permissive DSS method in PyMOL, Map True/False to a current list of human of PDB entries

Step 4. Matched segments are scored for structural similarly with a known degron structure (ZNF692 PDB 6H0G): Binet-Cauchy kernel score (BCscore) on Cα positions: (academic.oup.com/bioinformatics/article/30/6/784/286298); the BCscore is a cosine normalised similarity score: 1 is a perfect match, 0 for a completely dissimilar match, −1 for a mirror image.

Step 5. If the structural similarity for a match is ‘reasonable’ (BCscore>0.50) carry out further scoring: Clash score—superimpose onto a known a degron (ZNF692: PDB 6H0G) using backbone atoms) and calculate the atoms in the candidate neosubstrate model clashing with CRBN (closer than 1 Å)—return as an atom count and amino acid residue count. Surface accessibility calculation—a measure of structural isolation.

Results based on human Catalase are shown in Table 5.

TABLE 5

| | | | | | Similarity |
|---|---|---|---|---|---|
| PDB | Chain | Start | End | Seq | (BC score) |
| 1dgf | A | 41 | 46 | VITVGP | 0.8657 |
| 1dgf | A | 41 | 48 | VITVGPRG | 0.2162 |

Increasing the length of the search motif—from 6 aa to 8 aa—reduces the number of false positive G-loop hits.

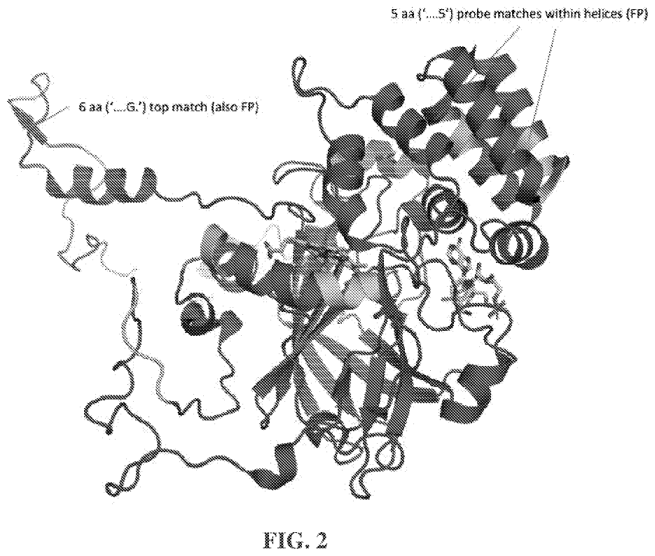

Decreasing the search motif—from 6 aa ('X¹—X²—X³—X⁴-G-X⁶) to 5 aa (‘X¹—X²—X³—X⁴-G’) results in false positive hits (motif is identified in the middle of alpha-helices). These false positives are avoided with a search motif with at least a 6 aa probe having a glycine at position number 5 (see FIG. 2).

### Example 2: Identification of Proteins Containing G-loops

Using the discovery process, approximately 842 putative G-loop containing proteins were identified. Validation of the discovery process is shown in Table 6.

As shown in WO 2021/069705, which is hereby incorporated by reference in its entirety, GSPT1 is bound by cereblon and selectively degraded in the presence of a number of E3 ligase binding modulators (see, e.g., pages 293-305).

TABLE 6
Analytics and general comments

| | Identified with | | | | | |
|---|---|---|---|---|---|---|
| Protein | discovery process | SASA** | clashes | BCscore** | RMSD | Sequence |
| CK1a | yes | 2.9 | 0 | 0.99 | 0.25 | Unique |
| GSPT1 | yes | 3.8 | 29-43 | 0.98 | 0.52 | Unique |
| IKZF1 | yes | 2.8 | 0 | 0.99 | 0.14 | Shared (IKZF4) |
| ZFP91 | yes | 2.4 | 62-323 | 0.99 | 0.42 | Unique |
| ZNF692 | yes | 2.8 | 0 | 0.99 | 0.04 | Unique |

*Unique entries, BC score >0.9, clashes <62, L-S-blank).
**SASA (measure of G-loop exposure/accessibility), Bcscore (3D similarity to the ZNF692 G-loop)

90% (756/842) of G-loops are unique in sequence.

Non-limiting examples of G-loop containing proteins identified are described in Table 7.

TABLE 7

| | | | | Druggable |
|---|---|---|---|---|
| Target | Target class | Cancer | Dependency* | by ligand** |
| GATA3 | Transcription | Breast | Yes | No |
| | factor (TF) | | | |
| NFE2L2 | TF | Lung | Yes | No |
| TRPS1 | TF | Breast | Yes | No |
| SPDEF | TF | Breast | Yes | No |
| TP63 | TF | Multiple | Yes | No |
| MECOM | TF | Ovarian | Yes | No |
| KLF5 | TF | CRC | Yes | No |
| SNAI2 | TF repressor | Multiple | Yes | No |
| LMO2 | TF complex | Hematology | Yes | No |
| ZBTB38 | Zinc Finger | Multiple | Yes | No |
| | (ZF) | myeloma | | |
| GFI1 | TF repressor | Hematology | Yes | No |

*Based on DepMap cancer cell dependency.
**Based on CanSAR assessment. Targets deemed undruggable.

### Example 3. Degradation Assay

To monitor protein degradation, the DiscoverX enzyme fragment contemplation assay (EFC) technology is used. The system relies on having two different components of the beta-galactosidase enzyme expressed for activity. A large protein fragment of β-galactoside is included in the InCELL Hunter detection reagent that is added at the end of the assay. The small peptide fragment (the enhanced ProLabel (ePL)) that is required for beta-galactosidase activity is expressed on the protein of interest (e.g., Aiolos and GSPT1). When the ePL tagged protein has been degraded through the E3-ligase mechanism, there is a loss in the β-galactosidase activity.

DF15 multiple myeloma cells stably expressing ePL-tagged Aiolos (or GSPT1) are generated via lentiviral infection with pLOC-ePL-Aiolos (or GSPT1). Cells are dispensed into a 384-well plate (Corning no. 3712) prespotted with compound. Compounds are dispensed by an acoustic dispenser (ATS acoustic transfer system from EDC Biosystems) into a 384-well in a 10 point dose-response curve using 3-fold dilutions starting at 10 μM and going down to 0.0005 μM in DMSO. A DMSO control is added to the assay. 0.25 μL of medium (RPMI-1640+10% heat inactivated FBS+25 mM Hepes+1 mM Na pyruvate+1×NEAA+0.1% Pluronic F-68+1×Pen Strep glutamine) containing 5000 cells is dispensed per well. Assay plates are incubated at 37° C. with 5% CO₂ for 4 h. After incubation, 25 μL of the InCELL Hunter detection reagent working solution (DiscoverX, catalog no. 96-0002, Fremont, CA) are added to each well and incubated at room temperature for 30 min protected from light. After 30 min, luminescence was read on a PHERAstar luminometer.

To determine EC₅₀ values for Aiolos or GSPT1 degradation, a four-parameter logistic model (sigmoidal dose-response model):

$$F I T = - A + \frac{B - A}{1 + {( \frac{C}{x} )}^{D}}$$

where C is the inflection point (EC₅₀), D is the correlation coefficient, and A and B are the low and high limits of the fit, respectively, was used to determine the compound's EC₅₀ value, which is the half-maximum effective concentration. The minimum Y is reference to the Y constant.

It is understood that the examples and embodiments described herein are for illustrative purposes only and that various modifications or changes in light thereof will be suggested to persons skilled in the art and are to be included within the spirit and purview of this application and scope of the appended claims.

NP_001166953.1
SEQ ID NO: 1
>NP_001166953.1 CRBN [organism = Homo sapiens] [GeneID = 51185]
[isoform = 2]
MAGEGDQQDAAHNMGNHLPLLPESEEEDEMEVEDQDSKEAKKPNIINFDTSLPTSHTYLGADMEEFHGRT
LHDDDSCQVIPVLPQVMMILIPGQTLPLQLFHPQEVSMVRNLIQKDRTFAVLAYSNVQEREAQFGTTAEI
YAYREEQDFGIEIVKVKAIGRQRFKVLELRTQSDGIQQAKVQILPECVLPSTMSAVQLESLNKCQIFPSK
PVSREDQCSYKWWQKYQKRKFHCANLTSWPRWLYSLYDAETLMDRIKKQLREWDENLKDDSLPSNPIDES
YRVAACLPIDDVLRIQLLKIGSAIQRLRCELDIMNKCTSLCCKQCQETEITTKNEIFSLSLCGPMAAYVN
PHGYVHETLTVYKACNLNLIGRPSTEHSWFPGYAWTVAQCKICASHIGWKFTATKKDMSPQKFWGLTRSA
LLPTIPDTEDEISPDKVILCL
NP_057386.2
SEQ ID NO: 2
>NP_057386.2 CRBN [organism = Homo sapiens] [GeneID = 51185] [isoform = 1]
MAGEGDQQDAAHNMGNHLPLLPAESEEEDEMEVEDQDSKEAKKPNIINFDTSLPTSHTYLGADMEEFHGR
TLHDDDSCQVIPVLPQVMMILIPGQTLPLQLFHPQEVSMVRNLIQKDRTFAVLAYSNVQEREAQFGTTAE
IYAYREEQDFGIEIVKVKAIGRQRFKVLELRTQSDGIQQAKVQILPECVLPSTMSAVQLESLNKCQIFPS
KPVSREDQCSYKWWQKYQKRKFHCANLTSWPRWLYSLYDAETLMDRIKKQLREWDENLKDDSLPSNPIDE
SYRVAACLPIDDVLRIQLLKIGSAIQRLRCELDIMNKCTSLCCKQCQETEITTKNEIFSLSLCGPMAAYV
NPHGYVHETLTVYKACNLNLIGRPSTEHSWFPGYAWTVAQCKICASHIGWKFTATKKDMSPQKFWGLTRS
ALLPTIPDTEDEISPDKVILCL
XP_005265259.1
SEQ ID NO: 3
>XP_005265259.1 CRBN [organism = Homo sapiens] [GeneID = 51185]
[isoform = X2]
MEEFHGRTLHDDDSCQVIPVLPQVMMILIPGQTLPLQLFHPQEVSMVRNLIQKDRTFAVLAYSNVQEREA
QFGTTAEIYAYREEQDFGIEIVKVKAIGRQRFKVLELRTQSDGIQQAKVQILPECVLPSTMSAVQLESLN
KCQIFPSKPVSREDQCSYKWWQKYQKRKFHCANLTSWPRWLYSLYDAETLMDRIKKQLREWDENLKDDSL
PSNPIDESYRVAACLPIDDVLRIQLLKIGSAIQRLRCELDIMNKCTSLCCKQCQETEITTKNEIFSLSLC
GPMAAYVNPHGYVHETLTVYKACNLNLIGRPSTEHSWFPGYAWTVAQCKICASHIGWKFTATKKDMSPQK
FWGLTRSALLPTIPDTEDEISPDKVILCL
XP_011532093.1
SEQ ID NO: 4
>XP_011532093.1 CRBN [organism = Homo sapiens] [GeneID = 51185]
[isoform = X1]
MAGEGDQQDAAHNMGNHLPLLPAESEEEDEMEVEDQDSKEAKKPNIINFDTSLPTSHTYLGADMEEFHGR
TLHDDDSCQVIPVLPQVMMILIPGQTLPLQLFHPQEVSMVRNLIQKDRTFAVLAYSNVQEREAQFGTTAE
IYAYREEQDFGIEIVKVKAIGRQRFKVLELRTQSDGIQQAKVQILPECVLPSTMSAVQLESLNKCQIFPS
KPVSREDQCSYKWWQKYQKRKFHCANLTSWPRWLYSLYDAETLMDRIKKQLREWDENLKDDSLPSNPIDE
SYRVAACLPIDDVLRIQLLKIGSAIQRLRCELDIMNKCTSLCCKQCQETEITTKNEIFRYAWTVAQCKIC
ASHIGWKFTATKKDMSPQKFWGLTRSALLPTIPDTEDEISPDKVILCL
XP_011532095.1
SEQ ID NO: 5
>XP_011532095. 1 CRBN [organism = Homo sapiens] [GeneID = 51185]
[isoform = x4]
MRLQHLLKMIFRIQQAKVQILPECVLPSTMSAVQLESLNKCQIFPSKPVSREDQCSYKWWQKYQKRKFHC
ANLTSWPRWLYSLYDAETLMDRIKKQLREWDENLKDDSLPSNPIDFSYRVAACLPIDDVLRIQLLKIGSA
IQRLRCELDIMNKCTSLCCKQCQETEITTKNEIFSLSLCGPMAAYVNPHGYVHETLTVYKACNLNLIGRP
STEHSWFPGYAWTVAQCKICASHIGWKFTATKKDMSPQKFWGLTRSALLPTIPDTEDEISPDKVILCL
XP_011532096.1
SEQ ID NO: 6
>XP_011532096.1 CRBN [organism = Homo sapiens] [GeneID = 51185]
[isoform = x4]
MRLQHLLKMIFRIQQAKVQILPECVLPSTMSAVQLESLNKCQIFPSKPVSREDQCSYKWWQKYQKRKFHC
ANLTSWPRWLYSLYDAETLMDRIKKQLREWDENLKDDSLPSNPIDFSYRVAACLPIDDVLRIQLLKIGSA
IQRLRCELDIMNKCTSLCCKQCQETEITTKNEIFSLSLCGPMAAYVNPHGYVHETLTVYKACNLNLIGRP
STEHSWFPGYAWTVAQCKICASHIGWKFTATKKDMSPQKFWGLTRSALLPTIPDTEDEISPDKVILCL
XP_024309319.1
SEQ ID NO: 7
>XP_024309319.1 CRBN [organism = Homo sapiens] [GeneID = 51185]
[isoform = X3]
MAGEGDQQDAAHNMGNHLPLLPAESEEEDEMEVEDQDSKEAKKPNIINFDTSLPTSHTYLGADMEEFHGR
TLHDDDSCQVIPVLPQVMMILIPGQTLPLQLFHPQEVSMVRNLIQKDRTFAVLAYSNVQEREAQFGTTAE
IYAYREEQDFGIEIVKVKAIGRQRFKVLELRTQSDGIQQAKVQILPECVLPSTMSAVQLESLNKCQIFPS
KPVSREDQCSYKWWQKYQKRKFHCANLTSWPRWLYSLYDAETLMDRIKKQLREWDENLKDDSLPSNPIVY
FPLL
ZNF692
SEQ ID NO: 8
>sp|Q9BU19|ZN692_HUMAN Zinc finger protein 692 OS = Homo sapiens
OX = 9606 GN = ZNF692 PE = 1 SV = 1
MASSPAVDVSCRRREKRRQLDARRSKCRIRLGGHMEQWCLLKERLGFSLHSQLAKELLDR
YTSSGCVLCAGPEPLPPKGLQYLVLLSHAHSRECSLVPGLRGPGGQDGGLVWECSAGHTE
SWGPSLSPTPSEAPKPASLPHTTRRSWCSEATSGQELADLESEHDERTQEARLPRRVGPP
PETFPPPGEEEGEEEEDNDEDEEEMLSDASLWTYSSSPDDSEPDAPRLLPSPVTCTPKEG
ETPPAPAALSSPLAVPALSASSLSSRAPPPAEVRVQPQLSRTPQAAQQTEALASTGSQAQ
SAPTPAWDEDTAQIGPKRIRKAAKRELMPCDFPGCGRIFSNRQYLNHHKKYQHIHQKSES
CPEPACGKSFNFKKHLKEHMKLHSDTRDYICEFCARSFRTSSNLVIHRRIHTGEKPLQCE
ICGFTCRQKASLNWHQRKHAETVAALRFPCEFCGKRFEKPDSVAAHRSKSHPALLLAPQE
SPSGPLEPCPSISAPGPLGSSEGSRPSASPQAPTLLPQQ
IKZF1
SEQ ID NO: 9
>sp|Q13422|IKZF1_HUMAN DNA-binding protein Ikaros OS = Homo
sapiens OX = 9606 GN = IKZF1 PE = 1 SV = 1
MDADEGQDMSQVSGKESPPVSDTPDEGDEPMPIPEDLSTTSGGQQSSKSDRVVASNVKVE
TQSDEENGRACEMNGEECAEDLRMLDASGEKMNGSHRDQGSSALSGVGGIRLPNGKLKCD
ICGIICIGPNVLMVHKRSHTGERPFQCNQCGASFTQKGNLLRHIKLHSGEKPFKCHLCNY
ACRRRDALTGHLRTHSVGKPHKCGYCGRSYKQRSSLEEHKERCHNYLESMGLPGTLYPVI
KEETNHSEMAEDLCKIGSERSLVLDRLASNVAKRKSSMPQKFLGDKGLSDTPYDSSASYE
KENEMMKSHVMDQAINNAINYLGAESLRPLVQTPPGGSEVVPVISPMYQLHKPLAEGTPR
SNHSAQDSAVENLLLLSKAKLVPSEREASPSNSCQDSTDTESNNEEQRSGLIYLTNHIAP
HARNGLSLKEEHRAYDLLRAASENSQDALRVVSTSGEQMKVYKCEHCRVLFLDHVMYTIH
MGCHG FRDPFECNMCGYHSQDRYEFSSHITRGEHRFHMS
SALL4
SEQ ID NO: 10
>sp|Q9UJQ4|SALL4_HUMAN Sal-like protein 4 OS = Homo sapiens
OX = 9606 GN = SALL4 PE = 1 SV = 1
MSRRKQAKPQHINSEEDQGEQQPQQQTPEFADAAPAAPAAGELGAPVNHPGNDEVASEDE
ATVKRLRREETHVCEKCCAEFFSISEFLEHKKNCTKNPPVLIMNDSEGPVPSEDESGAVL
SHQPTSPGSKDCHRENGGSSEDMKEKPDAESVVYLKTETALPPTPQDISYLAKGKVANTN
VTLQALRGTKVAVNQRSADALPAPVPGANSIPWVLEQILCLQQQQLQQIQLTEQIRIQVN
MWASHALHSSGAGADTLKTLGSHMSQQVSAAVALLSQKAGSQGLSLDALKQAKLPHANIP
SATSSLSPGLAPFTLKPDGTRVLPNVMSRLPSALLPQAPGSVLFQSPESTVALDTSKKGK
GKPPNISAVDVKPKDEAALYKHKCKYCSKVFGTDSSLQIHLRSHTGERPFVCSVCGHRFT
TKGNLKVHFHRHPQVKANPQLFAEFQDKVAAGNGIPYALSVPDPIDEPSLSLDSKPVLVT
TSVGLPQNLSSGTNPKDLTGGSLPGDLQPGPSPESEGGPTLPGVGPNYNSPRAGGFQGSG
TPEPGSETLKLQQLVENIDKATTDPNECLICHRVLSCQSSLKMHYRTHTGERPFQCKICG
RAFSTKGNLKTHLGVHRTNTSIKTQHSCPICQKKFTNAVMLQQHIRMHMGGQIPNTPLPE
NPCDFTGSEPMTVGENGSTGAICHDDVIESIDVEEVSSQEAPSSSSKVPTPLPSIHSASP
TLGFAMMASLDAPGKVGPAPENLQRQGSRENGSVESDGLINDSSSLMGDQEYQSRSPDIL
ETTSFQALSPANSQAESIKSKSPDAGSKAESSENSRTEMEGRSSLPSTFIRAPPTYVKVE
VPGTFVGPSTLSPGMTPLLAAQPRRQAKQHGCTRCGKNESSASALQIHERTHTGEKPFVC
NICGRAFTTKGNLKVHYMTHGANNNSARRGRKLAIENTMALLGTDGKRVSEIFPKEILAP
SVNVDPVVWNQYTSMLNGGLAVKTNEISVIQSGGVPTLPVSLGATSVVNNATVSKMDGSQ
SGISADVEKPSATDGVPKHQFPHELEENKIAVS
CKIalpha
SEQ ID NO: 11
>sp|P48729|KC1A_HUMAN Casein kinase I isoform alpha OS = Homo
sapiens OX = 9606 GN = CSNK1A1 PE = 1 SV = 2
MASSSGSKAEFIVGGKYKLVRKIGSGSFGDIYLAINITNGEEVAVKLESQKARHPQLLYE
SKLYKILQGGVGIPHIRWYGQEKDYNVLVMDLLGPSLEDLENFCSRRFTMKTVLMLADQM
ISRIEYVHTKNFIHRDIKPDNFLMGIGRHCNKLFLIDFGLAKKYRDNRTRQHIPYREDKN
LTGTARYASINAHLGIEQSRRDDMESLGYVLMYENRTSLPWQGLKAATKKQKYEKISEKK
MSTPVEVLCKGFPAEFAMYLNYCRGLRFEEAPDYMYLRQLFRILFRTLNHQYDYTEDWTM
LKQKAAQQAASSSGQGQQAQTPTGKQTDKTKSNMKGF
SEQ ID NO: 12
>sp|P15170|ERF3A_HUMAN Eukaryotic peptide chain release factor
GTP-binding subunit ERF3A OS = Homo sapiens OX = 9606 GN = GSPT1
PE = 1 SV = 1
MELSEPIVENGETEMSPEESWEHKEEISEAEPGGGSLGDGRPPEESAHEMMEEEEEIPKP
KSVVAPPGAPKKEHVNVVFIGHVDAGKSTIGGQIMYLTGMVDKRTLEKYEREAKEKNRET
WYLSWALDTNQEERDKGKTVEVGRAYFETEKKHFTILDAPGHKSFVPNMIGGASQADLAV
LVISARKGEFETGFEKGGQTREHAMLAKTAGVKHLIVLINKMDDPTVNWSNERYEECKEK
LVPFLKKVGENPKKDIHFMPCSGLTGANLKEQSDFCPWYIGLPFIPYLDNLPNENRSVDG
PIRLPIVDKYKDMGTVVLGKLESGSICKGQQLVMMPNKHNVEVLGILSDDVETDTVAPGE
NLKIRLKGIEEEEILPGFILCDPNNLCHSGRTFDAQIVIIEHKSIICPGYNAVLHIHTCI
EEVEITALICLVDKKSGEKSKTRPRFVKQDQVCIARLRTAGTICLETEKDFPQMGRETLR
DEGKTIAIGKVLKLVPEKD

### SEQUENCES

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of identifying a candidate substrate protein for cereblon, the method comprising:
(a) identifying a test protein comprising a test amino acid motif having the following formula:
X¹—X²—X³—X⁴—X⁵—Y;
wherein: Y is 1 to 10 amino acids of the formula X⁶, X⁶—X⁷, X⁶—X⁷—X⁸, X⁶—X⁷—X⁸—X⁹, X⁶—X⁷—X⁸—X⁹—X¹⁰, X⁶—X⁷—X⁸—X⁹—X¹⁰—X¹¹, X⁶—X⁷—X⁸—X⁹—X¹⁰—X¹¹—X¹², X⁶—X⁷—X⁸—X⁹—X¹⁰—X¹¹—X¹²—X¹³, X⁶—X⁷—X⁸—X⁹—X¹⁰—X¹¹—X²—X¹³—X¹⁴, or X⁶—X⁷—X⁸—X⁹—X¹⁰—X¹¹—X¹²—X¹³—X¹⁴—X¹⁵,
wherein each X is a single amino acid, and
wherein X⁵ is glycine, while each of the remaining amino acids are independently selected from any one of the natural occurring amino acids;
(b) identifying a corresponding reference amino acid motif from the protein sequence of a known substrate protein for cereblon, wherein the reference amino acid motif is of the same length in amino acids as the test amino acid motif, and wherein the reference amino acid motif has a glycine at amino acid position 5 within the motif;
(c) providing a three-dimensional structure for each of the test protein's amino acid motif and the reference amino acid motif;
(d) comparing the three-dimensional structure of the test amino acid motif and the reference amino acid motif;
(e) based on the comparison, classifying the test protein as a candidate substrate protein for cereblon or not; and
(f) optionally: determining one or more additional three-dimensional characterization score(s); and, based on the one or more additional three-dimensional characterization score(s), re-classifying the test protein as a candidate substrate protein for cereblon or not.

2. The method of claim 1, further comprising:
(g) testing the candidate substrate protein in an E3 ligase substrate detection assay or having the candidate substrate protein tested in an E3 ligase substrate detection assay.

3. The method of claim 1, wherein comparing the three-dimensional structure of the test protein's amino acid motif and the reference amino acid motif comprises:
(i) providing the three-dimensional coordinates of the Cα atoms for each amino acid in the test protein amino acid motif and for each amino acid in the reference amino acid motif;
(ii) calculating the Binet-Cauchy fragment similarity score (bc-score) between the test protein amino acid motif and the reference amino acid motif

4. The method of claim 3, wherein the test protein is classified as a candidate substrate protein for cereblon if the be-score is above 0.5, 0.55, 0.6, 0.65, 0.7, 0.75, 0.8, or 0.85.

5. The method of claim 1, wherein the known substrate protein for cereblon is selected from the group consisting of ZNF692, GSPT1, CK1alpha, IKZF1, ZNF692, and SALL4.

6. The method of claim 1, wherein providing the three-dimensional structure for the reference amino acid motif comprises providing a crystal structure selected from the group consisting of ZNF692 PDB 6H0G, GSPT1 PDB 5HXB, GSPT1 PDB 6XK6, CK1alpha PDB 5FQD, IKZF1 PDB 6H0F, ZNF692 PDB 6H0G, SALL4 PDB 6UML, SALL4 PDB 7BQV, or SALL4 PDB 7BQU.

7. The method of claim 1, wherein providing the three-dimensional structure for the reference amino acid motif comprises providing an AlphaFold2 structure selected from the group consisting of “Zinc finger protein 692, Q9BU19 (ZN692_HUMAN)”, “DNA-binding protein IKaros, Q13422 (IKZF1_HUMAN)”, “Sal-like protein 4, Q9UJQ4 (SALL4_HUMAN)”, “Casein kinase I isoform alpha, P48729, (KC1A_HUMAN)”, and “Eukaryotic peptide chain release factor GTP-binding subunit ERF3A, P15170, ERF3A_HUMAN”.

8. The method of claim 1, wherein the reference protein is ZNF692 and the reference amino acid motif begins at position 419 of Error! Reference source not found. and comprises 6, 7, 8, 9, 10, 11, 12, 13, 14, or 15 consecutive amino acids of Error! Reference source not found. (oriented N- to C- terminally from the beginning position), wherein the number of amino acids in the reference amino acid motif is the same as the number of amino acids in the test protein motif.

9. The method of claim 1, wherein the reference protein is IKZF1 and the reference amino acid motif begins at position 147 of Error! Reference source not found. and comprises 6, 7, 8, 9, 10, 11, 12, 13, 14, or 15 consecutive amino acids of Error! Reference source not found. (oriented N- to C- terminally from the beginning position), wherein the number of amino acids in the reference amino acid motif is the same as the number of amino acids in the test protein motif.

10. The method of claim 1, wherein the reference protein is SALL4 and the reference amino acid motif begins at position 412 of Error! Reference source not found. and comprises 6, 7, 8, 9, 10, 11, 12, 13, 14, or 15 consecutive amino acids of Error! Reference source not found. (oriented N- to C- terminally from the beginning position), wherein the number of amino acids in the reference amino acid motif is the same as the number of amino acids in the test protein motif.

11. The method of claim 1, wherein the reference protein is CK1alpha and the reference amino acid motif begins at position 36 of Error! Reference source not found. and comprises 6, 7, 8, 9, 10, 11, 12, 13, 14, or 15 consecutive amino acids of Error! Reference source not found. (oriented N- to C- terminally from the beginning position), wherein the number of amino acids in the reference amino acid motif is the same as the number of amino acids in the test protein motif.

12. The method of claim 1, wherein the reference protein is GSPT1 and the reference amino acid motif begins at position 433 of Error! Reference source not found. and comprises 6, 7, 8, 9, 10, 11, 12, 13, 14, or 15 consecutive amino acids of Error! Reference source not found. (oriented N- to C- terminally from the beginning position), wherein the number of amino acids in the reference amino acid motif is the same as the number of amino acids in the test protein motif.

13. The method of claim 1, wherein providing the three dimensional structure for the test protein comprises providing a crystal structure.

14. The method of claim 1, wherein providing the three dimensional structure for the test protein comprises providing a computer modelled three-dimensional structure.

15. The method of claim 1, wherein Y consists of X⁶.

16. The method of claim 1, wherein Y consists of X⁶—X⁷.

17. The method of claim 1, wherein the amino acid motif is at least 8 amino acids long.

18. The method of claim 1, wherein X¹ is aspartic acid (D) or asparagine (N); and wherein X⁴ is serine (S) or threonine (T).

19. The method of claim 1, wherein X¹ and X⁴ are the same.

20. The method of claim 18, wherein X¹ and X⁴ are both cysteine (C); or wherein X i and X⁴ are both asparagine (N).

21. The method of claim 2, wherein the E3 ligase substrate detection assay is carried out in the presence of an E3 ligase binding modulator.

22. The method of claim 1, wherein step (f) is not optional.

23. The method of claim 22, where the E3 ligase binding modulator is a targeted protein degrader.

24. The method of claim 1, wherein the one or more additional three-dimensional characterization score(s) are selected from the group consisting of structural context score(s), atomic distance score(s), cereblon binding compatibility score(s), surface accessibility score(s), geometry score(s), and combinations thereof.

25-48. (canceled)


SEQUENCE LISTING (12 sequences)

SEQ ID NO: 1 - protein, 441 aa, Homo sapiens
MAGEGDQQDAAHNMGNHLPLLPESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 2 - protein, 442 aa, Homo sapiens [sequence omitted]

SEQ ID NO: 3 - protein, 379 aa, Homo sapiens
MEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 4 - protein, 398 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFRYAWTVAQCKICASHIGWKFTATKKDMSPQKFWGLTRSALLPTIPDTEDEISPDKVILCL

SEQ ID NO: 5 - protein, 278 aa, Homo sapiens
MRLQHLLKMIFRIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 6 - protein, 278 aa, Homo sapiens; identical to SEQ ID NO: 5

SEQ ID NO: 7 - protein, 284 aa, Homo sapiens
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTMSAVQLESLNKCQIFPSKPVSREDQCSYKWWQKYQKRKFHCANLTSWPRWLYSLYDAETLMDRIKKQLREWDENLKDDSLPSNPIVYFPLL

SEQ ID NO: 8 - protein, 519 aa, Homo sapiens
MASSPAVDVSCRRREKRRQLDARRSKCRIRLGGHMEQWCLLKERLGFSLHSQLAKFLLDRYTSSGCVLCAGPEPLPPKGLQYLVLLSHAHSRECSLVPGLRGPGGQDGGLVWECSAGHTFSWGPSLSPTPSEAPKPASLPHTTRRSWCSEATSGQELADLESEHDERTQEARLPRRVGPPPETFPPPGEEEGEEEEDNDEDEEEMLSDASLWTYSSSPDDSEPDAPRLLPSPVTCTPKEGETPPAPAALSSPLAVPALSASSLSSRAPPPAEVRVQPQLSRTPQAAQQTEALASTGSQAQSAPTPAWDEDTAQIGPKRIRKAAKRELMPCDFPGCGRIFSNRQYLNHHKKYQHIHQKSFSCPEPACGKSFNFKKHLKEHMKLHSDTRDYICEFCARSFRTSSNLVIHRRIHTGEKPLQCEICGFTCRQKASLNWHQRKHAETVAALRFPCEFCGKRFEKPDSVAAHRSKSHPALLLAPQESPSGPLEPCPSISAPGPLGSSEGSRPSASPQAPTLLPQQ

SEQ ID NO: 9 - protein, 519 aa, Homo sapiens
MDADEGQDMSQVSGKESPPVSDTPDEGDEPMPIPEDLSTTSGGQQSSKSDRVVASNVKVETQSDEENGRACEMNGEECAEDLRMLDASGEKMNGSHRDQGSSALSGVGGIRLPNGKLKCDICGIICIGPNVLMVHKRSHTGERPFQCNQCGASFTQKGNLLRHIKLHSGEKPFKCHLCNYACRRRDALTGHLRTHSVGKPHKCGYCGRSYKQRSSLEEHKERCHNYLESMGLPGTLYPVIKEETNHSEMAEDLCKIGSERSLVLDRLASNVAKRKSSMPQKFLGDKGLSDTPYDSSASYEKENEMMKSHVMDQAINNAINYLGAESLRPLVQTPPGGSEVVPVISPMYQLHKPLAEGTPRSNHSAQDSAVENLLLLSKAKLVPSEREASPSNSCQDSTDTESNNEEQRSGLIYLTNHIAPHARNGLSLKEEHRAYDLLRAASENSQDALRVVSTSGEQMKVYKCEHCRVLFLDHVMYTIHMGCHGFRDPFECNMCGYHSQDRYEFSSHITRGEHRFHMS

SEQ ID NO: 10 - protein, 1053 aa, Homo sapiens
MSRRKQAKPQHINSEEDQGEQQPQQQTPEFADAAPAAPAAGELGAPVNHPGNDEVASEDEATVKRLRREETHVCEKCCAEFFSISEFLEHKKNCTKNPPVLIMNDSEGPVPSEDFSGAVLSHQPTSPGSKDCHRENGGSSEDMKEKPDAESVVYLKTETALPPTPQDISYLAKGKVANTNVTLQALRGTKVAVNQRSADALPAPVPGANSIPWVLEQILCLQQQQLQQIQLTEQIRIQVNMWASHALHSSGAGADTLKTLGSHMSQQVSAAVALLSQKAGSQGLSLDALKQAKLPHANIPSATSSLSPGLAPFTLKPDGTRVLPNVMSRLPSALLPQAPGSVLFQSPFSTVALDTSKKGKGKPPNISAVDVKPKDEAALYKHKCKYCSKVFGTDSSLQIHLRSHTGERPFVCSVCGHRFTTKGNLKVHFHRHPQVKANPQLFAEFQDKVAAGNGIPYALSVPDPIDEPSLSLDSKPVLVTTSVGLPQNLSSGTNPKDLTGGSLPGDLQPGPSPESEGGPTLPGVGPNYNSPRAGGFQGSGTPEPGSETLKLQQLVENIDKATTDPNECLICHRVLSCQSSLKMHYRTHTGERPFQCKICGRAFSTKGNLKTHLGVHRTNTSIKTQHSCPICQKKFTNAVMLQQHIRMHMGGQIPNTPLPENPCDFTGSEPMTVGENGSTGAICHDDVIESIDVEEVSSQEAPSSSSKVPTPLPSIHSASPTLGFAMMASLDAPGKVGPAPFNLQRQGSRENGSVESDGLTNDSSSLMGDQEYQSRSPDILETTSFQALSPANSQAESIKSKSPDAGSKAESSENSRTEMEGRSSLPSTFIRAPPTYVKVEVPGTFVGPSTLSPGMTPLLAAQPRRQAKQHGCTRCGKNFSSASALQIHERTHTGEKPFVCNICGRAFTTKGNLKVHYMTHGANNNSARRGRKLAIENTMALLGTDGKRVSEIFPKEILAPSVNVDPVVWNQYTSMLNGGLAVKTNEISVIQSGGVPTLPVSLGATSVVNNATVSKMDGSQSGISADVEKPSATDGVPKHQFPHFLEENKIAVS

SEQ ID NO: 11 - protein, 337 aa, Homo sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKQKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 12 - protein, 499 aa, Homo sapiens
MELSEPIVENGETEMSPEESWEHKEEISEAEPGGGSLGDGRPPEESAHEMMEEEEEIPKPKSVVAPPGAPKKEHVNVVFIGHVDAGKSTIGGQIMYLTGMVDKRTLEKYEREAKEKNRETWYLSWALDTNQEERDKGKTVEVGRAYFETEKKHFTILDAPGHKSFVPNMIGGASQADLAVLVISARKGEFETGFEKGGQTREHAMLAKTAGVKHLIVLINKMDDPTVNWSNERYEECKEKLVPFLKKVGFNPKKDIHFMPCSGLTGANLKEQSDFCPWYIGLPFIPYLDNLPNFNRSVDGPIRLPIVDKYKDMGTVVLGKLESGSICKGQQLVMMPNKHNVEVLGILSDDVETDTVAPGENLKIRLKGIEEEEILPGFILCDPNNLCHSGRTFDAQIVIIEHKSIICPGYNAVLHIHTCIEEVEITALICLVDKKSGEKSKTRPRFVKQDQVCIARLRTAGTICLETFKDFPQMGRFTLRDEGKTIAIGKVLKLVPEKD